(19) United States
(12) Patent Application Publication
(10) Pub. No.: US 2026/0294770 A1
(43) Pub. Date: Oct. 1, 2026

(54) FORMULATIONS OF NARINGENIN

(71) Applicant: DEBUT BIOTECHNOLOGY, INC., San Diego, CA (US)

(72) Inventor: Taylor Oswald, San Diego, CA (US)

(21) Appl. No.: 19/480,603

(22) PCT Filed: May 9, 2024
(86) PCT No.: PCT/US2024/028524

§ 371 Date: Oct. 31, 2025

Related U.S. Application Data
(60) Provisional application No. 63/465,383, filed on May 10, 2023.
(60) Provisional application No. 63/465,381, filed on May 10, 2023.
(60) Provisional application No. 63/564,366, filed on Mar. 12, 2024.

(51) Int. Cl. A61K 8/49; A61Q 17/00; A61Q 19/08

(52) U.S. Cl. CPC A61K 8/498; A61Q 17/00; A61Q 19/08; A61K 2800/49; A61K 2800/51; A61K 2800/524; A61K 2800/56; A61K 2800/782

(57) ABSTRACT

The invention provides compositions comprising naringenin. The compositions of the invention may be administered topically, on the skin of the subject. The invention further provides methods of administration of compositions comprising naringenin for amelioration or treatment of a skin condition. The compositions of the invention may also be applied for improving the general appearance of the skin.

### I. FIELD OF THE INVENTION

The invention is related to formulations of naringenin and methods of using the formulations for topical conditions in a subject.

### II. BACKGROUND OF THE INVENTION

Mammalian epidermis and its appendages (e.g., hair, nail, sebaceous and sweat glands) provide a barrier to keep harmful elements out of the body and essential body fluids in. As the first line of defense against the various physical traumas of the environment, the epidermis must protect itself as well as the underlying tissues. The epidermis is also exposed to mutagenic ultraviolet radiation. In nonhaired or sparsely haired regions such as most human skin, the epidermis is thicker than that of furred skin, and in these locations the skin functions primarily in a protective role. The constant assaults on the epidermis necessitate self-renewal, making the epidermis a prime example of an adult tissue that undergoes continual and rapid flux.

Aged skin differs from youthful skin both in appearance and in function. The aged epidermis demonstrates decreased keratinocyte proliferation and is physically thinner, but mostly from decline of the rete ridges. In addition to thinning, aging slows wound healing, prolongs epidermal turnover, and impairs barrier formation. The skin appears thin, wrinkled, bruised, and rough. Wrinkling and bruising can also result from aging-related changes in the dermis. The dermis, too, is characteristically thinner in aged persons. Aged fibroblasts are less likely to synthesize normal amounts of collagen, elastin, laminin glycosaminoglycans, and fibronectin. The dermis can, in such cases, lack elasticity, strength, vessel support, remodeling abilities, and ground substances. For example, rete ridges can be effaced, and basal cells can no longer display villous projections into the dermis. Epidermal cell turnover is reduced up to 50% in the aged as compared to youth.

For example, melanocyte numbers can decrease 8-20% per decade. There are fewer Langerhans cells in the aged, and those present are often functionally impaired. Collagen synthesis decreases, for example, up to 30% within 4 years of menopause in women. The numbers of collagen and elastic fibers are also decreased. The dermis can also become less echogenic to ultrasounds, consistent with changes in collagen and elastic tissues.

### III. SUMMARY OF THE INVENTION

The invention relates to compositions of naringenin, and methods of using the compositions for treating, protecting, and/or altering (e.g., improving) the condition and/or aesthetic appearance of skin, including, for example, treating, preventing, ameliorating, reducing and/or eliminating fine lines and/or wrinkles of skin and/or improving the aesthetic appearance of fine lines and/or wrinkles of skin. In certain embodiments, the compositions of the invention lead to increase in skin and/or hair resiliency. In certain embodiments, the compositions of the invention lead to increase in elasticity of the skin. In certain embodiments, the compositions of the invention prevent skin damage.

The use of compositions comprising naringenin for skin care was not widely used in the field of skin care. The invention recognizes that compositions comprising naringenin may be used for development of compositions that may have a beneficial impact on the skin. In particular, the topical administration of naringenin for improving the condition of skin, including enhancing the appearance of the skin, by topical administration of a composition comprising an effective amount of naringenin was not known in the field. Beneficially, the invention recognizes, for the first time, that topical compositions comprising naringenin may be used for improving the condition of the skin or for treatment of any ailment related to the skin of a subject. In one aspect, the invention includes a composition for topical administration comprising an effective amount of naringenin. The effective amount of naringenin in a composition is an amount of naringenin that would be effective in treating, ameliorating, or otherwise impacting the underlying condition of the skin. The invention recognizes that naringenin is poorly soluble or insoluble in water. The invention also recognizes that naringenin is also poorly soluble or insoluble in oil, for example, naringenin is insoluble in Helianthus Annuus (Sunflower) Seed Oil. In certain embodiments of the invention, naringenin is dissolved in a solvent selected from a group consisting of: dimethyl isosorbide, ethoxydiglycol, isopropyl lauryl sarcosinate, pentylene glycol, propanediol, 1,2-hexanediol, and/or butylene glycol. In certain preferred embodiments, naringenin is dissolved in dimethyl isosorbide. In certain embodiments, naringenin is dissolved in isopropyl lauryl sarcosinate. In certain embodiments, naringenin is dissolved in ethoxydiglycol. In certain embodiments, the solvent is a glycol. In certain embodiments, the glycol is selected from a group consisting of pentylene glycol, butylene glycol, and any combination thereof. In certain embodiments, the solvent is selected from a group consisting of: phenylpropanol, 1,2-hexanediol, propanediol, isopentyldiol, 1,2 heptanediol, and any combination thereof.

In certain embodiments, the solvents are selected from a group consisting of: 1,2 heptanediol, 1,2 hexanediol, 1,2 propanediol, butylene glycol, C₁₅₋₁₉ alkanes, capric/caprylic triglyceride, dibutyl adipate, diisopropyl adipate, dimethyl isosorbide, dipropylene glycol, ethanol, ethoxydiglycol, isoamyl laurate, isohexadecane, isopentyldiol, isopropyl myristate, isopropyl lauryl sarcosinate, jojoba oil, meadowfoam seed oil, mineral oil, neopentyl glycol diethylhexanoate, octyldodecanol, oleyl alcohol, pentylene glycol, phenylpropanol, polysorbate 20, propanediol, safflower seed oil, squalene, sunflower oil, t-butyl alcohol, tributyl citrate, tributyl-O-acetylcitrate, Tri-C₁₅₋₁₉ alkyl citrate, triethyl citrate, triethyl O-acetylcitrate, and triisostearin.

In certain embodiments, the composition comprising naringenin is anhydrous. In certain embodiments, the composition comprises oil as the only solvent. In certain embodiments, the composition comprising naringenin is an emulsion. In certain embodiments, the composition comprising naringenin is a microemulsion. In certain embodiments, the compositions of the invention are encapsulated. In certain embodiments, the naringenin may be microencapsulated.

In another aspect, the invention is directed to stable compositions of naringenin. In certain embodiments, the compositions of the invention are stable under storage conditions. In certain embodiments, the compositions comprising naringenin are stable under storage in ambient conditions for a period of at least 2 years. In certain embodiments, the compositions comprising naringenin are stable under storage in ambient conditions for a period of at least 1 year. In certain embodiments, the compositions comprising naringenin are stable under storage in ambient conditions for a period of at least six (6) months. In certain embodiments, the compositions comprising naringenin are stable under storage in ambient conditions for a period of at least three (3) months.

The stability studies conducted with naringenin dissolved in dimethyl isosorbide, isopropyl sarcosinate, and ethoxydiglycol demonstrate that naringenin has less than 10% degradation when stored under accelerated stability conditions for a duration of one (1) month. These results demonstrate that the compositions of the invention would be stable for a duration of at least two (2) years under ambient conditions. In certain embodiments, the ambient conditions mean that the compositions are stable when stored at room temperature (in the range of about 15° C. to about 25° C.).

In certain embodiments, the composition comprises naringenin in a concentration of about 5 nM to about 2500 nM. In certain embodiments, the composition comprises naringenin in a concentration of about 10 nM to about 2000 nM. In certain embodiments, the composition comprises naringenin in a concentration of about 20 nM nM to about 1000 nM. In certain embodiments, the composition comprises naringenin in a concentration of about 10 nM to about 20 nM.

In certain embodiments, in the compositions of the invention, naringenin is present in a concentration of about 0.0001% to about 5.0% w/w. In certain embodiments, in the compositions of the invention, naringenin is present in a concentration of about 0.0001% to about 1.0% w/w. In certain embodiments, in the compositions of the invention, naringenin is present in a concentration of about 0.001% to about 0.5% w/w. In certain embodiments, in the compositions of the invention, naringenin is present in a concentration of about 0.01% to about 0.5% w/w. In certain embodiments, in the compositions of the invention, naringenin is present in a concentration of about 0.1% to about 0.5% w/w. In certain embodiments, in the compositions of the invention, naringenin is present in a concentration of about 0.45% w/w.

In certain aspects, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment. In certain embodiments, the compositions of the invention also include excipients. These excipients may be selected from a group consisting of: solvent, emulsifier, preservative, antioxidant, emollient, thickening agent, penetration enhancer, surfactant, diluent, filler, carrier, and/or pH control agent. In certain embodiments, the preservative in the composition is an antimicrobial preservative or a chelating agent. In certain embodiments, the solvent of the invention is dimethyl isosorbide. In certain embodiments, the solvent of the invention is isopropyl lauryl sarcosinate. In certain embodiments, the solvent of the invention is ethoxydiglycol.

The compositions of the invention may be prepared by various methods. As an example, the compositions may be prepared by dissolving, dispersing, etc. naringenin as provided herein and optional excipients in a carrier (e.g., water, saline, aqueous dextrose, glycerol, glycols, ethanol or the like) or a solvent to form a solution, colloid, liposome, emulsion, complexes (including inclusion complexes), coacervate, or suspension. In certain embodiments, the compositions of the current invention can also contain additional excipients such as wetting agents, emulsifying agents, co-solvents, solubilizing agents, pH buffering agents, and the like (e.g., sodium acetate, sodium citrate, cyclodextrins and derivatives, sorbitan monolaurate, triethanolamine acetate, triethanolamine oleate, hydroxyethylpiperazine and the like). In certain embodiments, the compositions of the invention may further include one or more additional excipients selected from a group consisting of: carriers, excipients, or diluents including, but not limited to, absorbents, anti-irritants, antibacterials, anti-acne agents, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In certain embodiments, the compositions of the invention comprise: naringenin, in an oil/water emulsion, and a preservative.

In certain embodiments, the compositions of the invention comprise: naringenin, dissolved in a solvent, and a preservative.

In certain embodiments, the compositions of the invention comprise: naringenin, in an oil/water emulsion, a solvent for solubilizing naringenin, and a preservative.

In certain embodiments, the compositions of the invention comprise naringenin, a solvent for solubilizing naringenin, a diluent, and a preservative.

In certain embodiments, the compositions of the invention comprise: naringenin, a solvent for solubilizing naringenin, a thickener, optionally a diluent, and a preservative.

In certain embodiments, the compositions of the invention comprise: naringenin, a solvent for solubilizing naringenin, and one or more excipients selected from the group consisting of: diluents, absorbents, antioxidants, emollients, emulsifiers, thickeners, surfactants, and/or preservatives.

In certain embodiments, the excipients included in the composition are water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and/or ethylhexylglycerin.

In certain aspects, the invention provides that the compositions of the inventions are synergistic. The compositions provided in the invention are beneficial because of their enhanced stability due to the synergistic effects of the ingredients in the composition and the resulting stability and efficacy. In certain embodiments, the invention provides that the compositions of the invention are more efficacious and stable as compared to when they are administered or stored as individual ingredients. The compositions of the invention may also act synergistically when administered along with other therapies for treatment of the conditions described herein.

In certain aspects of the invention, the compositions comprising naringenin demonstrate antioxidant activity. In certain embodiments, the compositions of the invention, the antioxidant activity of naringenin is demonstrated at concentrations of about 0.0001% w/w or higher. In certain embodiments, the compositions of the invention, the antioxidant activity of naringenin has activity at a concentration of about 0.0005% w/w or higher.

Advantageously, the compositions of the invention comprising naringenin, demonstrating antioxidant activity, are effective for treating and/or preventing oxidation damage. In certain embodiments, the compositions of the invention comprising naringenin, demonstrating antioxidant activity, are effective for treatment of skin and/or hair damaged by reactive oxygen species and protection from other environmental aggressors that may damage the skin of the subject. In certain other embodiments, the compositions of the invention comprising naringenin, demonstrating antioxidant activity, are effective for prevention of skin and/or hair damage by reactive oxygen species, including the damage caused by environmental aggressors.

In certain aspects of the invention, the compositions comprising naringenin demonstrate elastase enzyme inhibition. In certain embodiments, the compositions of the invention, the elastase enzyme inhibition activity of naringenin is demonstrated at concentrations of about 0.02% w/w or higher. In certain embodiments, the compositions of the invention, the elastase enzyme inhibition activity of naringenin is demonstrated at concentrations of about 0.1% w/w or higher. Advantageously, the compositions comprising naringenin, demonstrating elastase enzyme inhibition activity, are effective for promoting the elasticity of the skin. In certain embodiments, the compositions of the invention comprising naringenin, demonstrating elastase enzyme inhibition, are effective in reduction of fine lines and/or wrinkles in the skin. In certain embodiments, the compositions of the invention comprising naringenin, demonstrating elastase enzyme inhibition, are effective in prevention of formation of fine lines and/or wrinkles in the skin.

In certain aspects of the invention, the compositions comprising naringenin demonstrate inhibition of glycation. In certain embodiments, the compositions of the invention, the glycation inhibition activity of naringenin is demonstrated at concentrations of about 0.005% w/w or higher. In certain embodiments, the compositions of the invention, the glycation inhibition activity of naringenin is demonstrated at concentrations of about 0.008% w/w or higher. Advantageously, the compositions comprising naringenin, demonstrating glycation inhibition activity, are effective for supporting and promoting skin and/or hair resiliency.

In certain aspects of the invention, the compositions comprising naringenin demonstrate anti-inflammatory activity. In certain embodiments, the compositions comprising naringenin demonstrate anti-inflammatory activity at a concentration of 0.0001% w/w or higher. In certain embodiments, the compositions comprising naringenin demonstrate anti-inflammatory activity at a concentration of 0.0045% w/w or higher. In certain embodiments, the anti-inflammatory activity of naringenin in the compositions of the invention is demonstrated by reduction in anti-inflammatory markers IL-6 and/or TNF-α in stressed keratinocytes. In certain embodiments, the anti-inflammatory activity is demonstrated in human epidermal keratinocytes. The chemokines produced by activated keratinocytes and immune cells are able to damage the skin tissue in proximity to the target of the inflammatory response. Modulation of immune response is important in maintaining skin homeostasis. If the modulation is functioning optimally, the inflammatory response of the cells would disrupt skin homeostasis. Advantageously, the anti-inflammatory activity of compositions comprising naringenin is beneficial in maintaining this skin homeostasis and promoting the health of the skin.

In certain aspects of the invention, the composition is a composition comprising naringenin that can be applied as a mask on the skin. In certain embodiments, the mask may be for overnight application. Accordingly, in certain embodiments, the application of the mask results in refreshing appearance of the skin.

In certain aspects, the invention provides sunscreens comprising naringenin. In certain embodiments, the sunscreens comprising naringenin provide full broad spectrum light protection and repair on inflammaging damage to sustain firmness.

In certain aspects, the invention provides fortifying eye cream comprising naringenin. In certain embodiments, the eye cream firms and hydrates the skin to reduce visible signs of inflammaging.

In certain aspects of the invention, a composition comprising naringenin modulates expression of one more genes that are related to a topical condition. In certain embodiments, administration of composition comprising naringenin alters one or more of a function related with the modulated genes, wherein the one or more function is selected from the group consisting of: (i) altering the appearance of the skin or ameliorating a condition leading to undesired appearance of the skin, (ii) formation of fines lines or wrinkles on the skin, (iii) inflammation, (iv) barrier support maintenance, and (v) altering cell turnover or cell recycling to change the appearance of the skin. In certain embodiments, administration of composition comprising naringenin alters expression and/or function of one or more genes are selected from a group consisting of: LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, MIR205, CCL11, CSF2, IL23A, IL11, BMP6, IL3RA2, C2CD4A, NR4A2, C2CD4B, FGF2, FABP4, GDF15, THBS1, HSPA1A, PTGS2, and/or DKK1.

In certain embodiments, administration of a composition comprising naringenin leads to up-regulation of LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, and/or MIR205. In certain embodiments, administration of a composition comprising naringenin leads to down-regulation of CCL11, CSF2, IL23A, IL11, BMP6, IL13RA2, C2CD4A, NR4A2, C2CD4B, FGF2, FABP4, GDF15, THBS1, HSPA1A, PTGS2, and/or DKK1.

In certain aspects of the invention, administration of a composition comprising naringenin enhances anti-inflammatory activity. In certain embodiments, administration of a composition comprising naringenin enhances anti-inflammatory activity because it leads to downregulation of CSF2, IL23A, IL11, IL13RA2, C2CD4A, NR4A2, FABP4, GDF15, and/or PTGS2. In certain embodiments, administration of a composition comprising naringenin enhances anti-inflammatory activity because it leads to upregulation of HPGD. In certain embodiments, the administration of composition comprising naringenin results in at least about fifteen (15) fold downregulation of expression and/or activity of CSF2 In certain embodiments, the administration of composition comprising naringenin results in at least about fifteen (15) fold downregulation of expression and/or activity of IL23A. In certain embodiments, the administration of composition comprising naringenin results in at least about ten (10) fold downregulation of expression and/or activity of IL11. In certain embodiments, the administration of composition comprising naringenin results in at least about ten (10) fold downregulation of expression and/or activity of IL13RA2. In certain embodiments, the administration of composition comprising naringenin results in at least about ten (10) fold downregulation of expression and/or activity of C2CD4A. In certain embodiments, the administration of composition comprising naringenin results in at least about ten (10) fold downregulation of expression and/or activity of NR4A2. In certain embodiments, the administration of composition comprising naringenin results in at least about five (5) fold downregulation of expression and/or activity of FABP4. In certain embodiments, the administration of composition comprising naringenin results in at least about five (5) fold downregulation of expression and/or activity of GDF15. In certain embodiments, the administration of composition comprising naringenin results in at least about five (5) fold downregulation of expression and/or activity of PTGS2. In certain embodiments, the administration of composition comprising naringenin results in at least about ten (10) fold up-regulation in expression and/or activity of HPGD.

In certain aspects of the invention, administration of a composition comprising naringenin enhances the skin barrier strength and/or maintenance of skin barrier lipids. In certain embodiments, administration of a composition comprising naringenin enhances the skin barrier strength and/or maintenance of skin barrier lipids because it leads to upregulation of LCE5A, KRT77, ALOXE3, KRT1, CASP14, KRT10, and/or LCE2B. In certain embodiments, the administration of composition comprising naringenin results in at least about thirty (30) fold up-regulation in expression and/or activity of LCE5A. In certain embodiments, the administration of composition comprising naringenin results in at least about ten (10) fold up-regulation in expression and/or activity of KRT77. In certain embodiments, the administration of composition comprising naringenin results in at least about ten (10) fold up-regulation in expression and/or activity of ALOXE3. In certain embodiments, the administration of composition comprising naringenin results in at least about five (5) fold up-regulation in expression and/or activity of KRT1. In certain embodiments, the administration of composition comprising naringenin results in at least about ten (10) fold up-regulation in expression and/or activity of CASP14. In certain embodiments, the administration of composition comprising naringenin results in at least about five (5) fold up-regulation in expression and/or activity of KRT10. In certain embodiments, the administration of composition comprising naringenin results in at least about five (5) fold up-regulation in expression and/or activity of LCE2B.

In certain aspects of the invention, administration of a composition comprising naringenin leads to enhancement of cell turnover and/or cell recycling. Beneficially, this enhancement of cell turnover and/or cell recycling leads to an antiaging effect. In certain embodiments, the antiaging effect may be demonstrated by reduction and/or prevention of formation of fine lines and/or wrinkles in the skin. In certain embodiments, administration of a composition comprising naringenin enhances the cell turnover and/or cell recycling in the skin because the administration of the composition leads to upregulation of DNASE1L2, SERPINB12, UPK1A, CALML5 and/or MIR205. In certain embodiments, administration of a composition comprising naringenin enhances the cell turnover and/or cell recycling in the skin because the administration of the composition leads to downregulation of BMP6 and/or HSPA1A. In certain embodiments, the administration of composition comprising naringenin results in at least about five (5) fold up-regulation in expression and/or activity of DNASE1L2. In certain embodiments, the administration of composition comprising naringenin results in at least about ten (10) fold up-regulation in expression and/or activity of SERPINB12. In certain embodiments, the administration of composition comprising naringenin results in at least about ten (10) fold up-regulation in expression and/or activity of UPK1A. In certain embodiments, the administration of composition comprising naringenin results in at least about five (5) fold up-regulation in expression and/or activity of CALML5. In certain embodiments, the administration of composition comprising naringenin results in at least about five (5) fold up-regulation in expression and/or activity of MIR205. In certain embodiments, the administration of composition comprising naringenin results in at least about ten (10) fold downregulation of expression and/or activity of BMP6. In certain embodiments, the administration of composition comprising naringenin results in at least about five (5) fold downregulation of expression and/or activity of HSPA1A.

In certain embodiments, administration of a composition comprising naringenin to a subject leads to the treatment or prevention of a topical condition of the subject through the modulation of levels of expression or activity of one or genes that may be involved in the topical condition. In certain embodiments, administration of a composition comprising naringenin alters one or more of a function related with the modulated genes, wherein the one or more function is selected from the group consisting of: (i) cell turnover, (ii) inflammation, (iii) barrier support, and (iv) any combination thereof.

### IV. BRIEF DESCRIPTION OF THE DRAWINGS

FIG. 1 provides the data pertaining to application of compositions comprising naringenin to the skin.

### V. DETAILED DESCRIPTION

### Naringenin

Naringenin is a flavonoid commonly found in a variety of citrus fruits. Naringenin is also referred to as: (2S)-5,7-Dihydroxy-2-(4-hydroxyphenyl)-2,3-dihydro-4H-1-benzopyran-4-one, (2S)-4′,5,7-Trihydroxyflavan-4-one, naringetol, salipurol, or salipurpol. The chemical structure of naringenin is provided below:

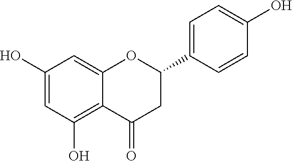 [SMILES: O=C1C[C@@H](c2ccc(O)cc2)Oc2cc(O)cc(O)c21]

Naringenin has demonstrated numerous biological activities, including anti-inflammatory properties, antioxidant activity, pain management, wound healing, and attenuation of fatty liver disease. Rebello C j et al. Safety and pharmacokinetics of naringenin: A randomized, controlled, single-ascending-dose clinical trial. Diabetes Obes Metab. 2020 (1):91-98; Martinez R M et al. Naringenin Inhibits UVB Irradiation-Induced Inflammation and Oxidative Stress in the Skin of Hairless Mice. J Nat Prod. 2015 Jul. 24; 78(7):1647-55; Pinho-Ribeiro F A, et al. Naringenin reduces inflammatory pain in mice. Neuropharmacology. 2016, 105:508-519; Wang Q, et al. Naringenin attenuates non-alcoholic fatty liver disease by down-regulating the NLRP3/NF-κB pathway in mice. Br J Pharmacol. 2020 April; 177(8):1806-1821.

Naringenin is generally considered safe for oral ingestion and shown to increase antioxidant markers superoxide dismutase and glutathione when consumed orally. Al-Roujayee A S. Naringenin improves the healing process of thermally-induced skin damage in rats. J Int Med Res. 2017 April; 45(2):570-582. In mouse models intraperitoneal naringenin treatment effectively inhibits edema, lowers cytokine production, inhibits UVB-induced skin damage, and lowers symptoms associated with atopic dermatitis. Karuppagounder Vet al. Naringenin ameliorates skin inflammation and accelerates phenotypic reprogramming from M1 to M2 macrophage polarization in atopic dermatitis NC/Nga mouse model. Exp Dermatol. 2016 May; 25(5):404-7; Kim T H et al. The inhibitory effect of naringenin on atopic dermatitis induced by DNFB in NC/Nga mice. Life Sci. 2013 Oct. 10; 93(15):516-24; Martinez R M, et al. Topical Formulation Containing Naringenin: Efficacy against Ultraviolet B Irradiation-Induced Skin Inflammation and Oxidative Stress in Mice. PLoS One. 2016, 11(1): e0146296; Pinho-Ribeiro Faet al. The citrus flavanone naringenin reduces lipopolysaccharide-induced inflammatory pain and leukocyte recruitment by inhibiting NF-κB activation. J Nutr Biochem. 2016 July 33:8-14.

Further, topical application in wounded mice with a naringenin- and alginate hydrogel resulted in more rapid wound closure. In vitro models suggest similar modes of action in human skin cells, with 10 μM doses protecting HaCaT immortalized keratinocytes from UVB-induced apoptosis while separate studies have shown restoration of skin barrier in psoriasis models. El-Mahdy M A et al. Naringenin protects HaCaT human keratinocytes against UVB-induced apoptosis and enhances the removal of cyclobutane pyrimidine dimers from the genome. Photochem Photobiol. 2008 March-April; 84(2):307-16; Alalaiwe A, et al. Development of flavanone and its derivatives as topical agents against psoriasis: The prediction of therapeutic efficiency through skin permeation evaluation and cell-based assay. Int J Pharm. 2020 May 15, 581:119256.

However, the use of compositions comprising an effective amount of naringenin for topical application to improve topical conditions has not been demonstrated before. The invention provides compositions comprising naringenin for administration on the skin of humans. In one aspect, the invention provides the use of compositions comprising naringenin in a topical cosmetic formulation as an anti-inflammatory with barrier restoration and antioxidant properties.

### Compositions of Naringenin

In one aspect, the invention provides compositions comprising naringenin. In certain embodiments, the invention provides compositions comprising naringenin that are suitable for administration on the skin of the subject.

The invention recognizes that naringenin is poorly soluble or insoluble in water. The invention also recognizes that naringenin is also poorly soluble or insoluble in oil, for example, naringenin is insoluble in Helianthus Annuus (Sunflower) Seed Oil. The invention recognizes that the solvent used for preparing naringenin compositions should be any solvent acceptable to be used in cosmetic formulations. In certain embodiments of the invention, naringenin is dissolved in a solvent selected from a group consisting of: dimethyl isosorbide, ethoxydiglycol, isopropyl lauryl sarcosinate, pentylene glycol, propanediol, 1,2-hexanediol, and/or butylene glycol. In certain preferred embodiments, naringenin is dissolved in dimethyl isosorbide. In certain embodiments, naringenin is dissolved in isopropyl lauryl sarcosinate. In certain embodiments, naringenin is dissolved in ethoxydiglycol. In certain embodiments, the solvent is a glycol. In certain embodiments, the glycol is selected from a group consisting of pentylene glycol, butylene glycol, and any combination thereof. In certain embodiments, the solvent is selected from a group consisting of: phenylpropanol, 1,2-hexanediol, propanediol, isopentyldiol, 1,2 heptanediol, and any combination thereof.

In certain embodiments, the solvents are selected from a group consisting of: 1,2 heptanediol, 1,2 hexanediol, 1,2 propanediol, butylene glycol, C₁₅₋₁₉ alkanes, capric/caprylic triglyceride, dibutyl adipate, diisopropyl adipate, dimethyl isosorbide, dipropylene glycol, ethanol, ethoxydiglycol, isoamyl laurate, isohexadecane, isopentyldiol, isopropyl myristate, isopropyl lauryl sarcosinate, jojoba oil, meadowfoam seed oil, mineral oil, neopentyl glycol diethylhexanoate, octyldodecanol, oleyl alcohol, pentylene glycol, phenylpropanol, polysorbate 20, propanediol, safflower seed oil, squalene, sunflower oil, t-butyl alcohol, tributyl citrate, tributyl-O-acetylcitrate, Tri-C₁₅₋₁₉ alkyl citrate, triethyl citrate, triethyl O-acetylcitrate, and triisostearin.

Table 1 provides the solubility data for naringenin in several solvents.

TABLE 1
Solubility of Naringenin in various solvents:

| Solvent | Naringenin Solubility (% w/w) |
|---|---|
| Ethoxydiglycol | 20.00 |
| Dimethyl isosorbide | 15.00 |
| Phenylpropanol | 5.00 |
| 1,2 hexanediol | 2.50 |
| Propanediol | 2.50 |
| Butylene glycol | 2.50 |
| Pentylene glycol | 2.50 |
| Isopentyldiol | 2.50 |
| 1,2 heptanediol | 2.50 |
| 3-methyl-1,3-butanediol | 0.00 |
| Oleyl alcohol | 0.00 |
| 1,2 propanediol | 0.00 |
| Capric/caprylic | 0.00 |
| triglyceride | |
| Di(propylene glycol) | 0.00 |
| dibenzoate | |
| Isoamyl laurate | 0.00 |
| Isopropyl myristate | 0.00 |
| Neopentyl glycol | 0.00 |
| diethylhexanoate | |
| Tri C12-C13 alkyl citrate | 0.00 |
| Tributyl citrate | 0.00 |
| Tributyl-O-acetylcitrate | 0.00 |
| Triethyl citrate | 0.00 |
| Triethyl O-acetylcitrate | 0.00 |
| Triisostearin | 0.00 |
| Isopropyl lauryol | 10.00 |
| sarcosinate | |
| C15-19 alkane | 0.00 |
| C9-12 alkane | 0.00 |
| jojoba oil | 0.00 |
| Squalene | 0.00 |
| sunflower oil | 0.00 |

In certain embodiments, the composition comprising naringenin is anhydrous. In certain embodiments, the composition comprises oil as the only solvent. In certain embodiments, the composition comprising naringenin is an emulsion. In certain embodiments, the composition comprising naringenin is a microemulsion. In certain embodiments, the compositions of the invention are encapsulated. In certain embodiments, the naringenin may be microencapsulated.

In another aspect, the invention is directed to stable compositions of naringenin. In certain embodiments, the compositions of the invention are stable under storage conditions. In certain embodiments, the compositions comprising naringenin are stable under storage in ambient conditions for a period of at least 2 years. In certain embodiments, the compositions comprising naringenin are stable under storage in ambient conditions for a period of at least 1 year. In certain embodiments, the compositions comprising naringenin are stable under storage in ambient conditions for a period of at least six (6) months. In certain embodiments, the compositions comprising naringenin are stable under storage in ambient conditions for a period of at least three (3) months. The invention beneficially provides that solutions of naringenin dissolved in dimethyl isosorbide, isopropyl lauroyl sarcosinate, and ethoxydiglycol have increased solubility and are shelf stable. Since naringenin is not soluble in sunflower oil and other commonly used solvents, the increased solubility in dimethyl isosorbide, isopropyl lauroyl sarcosinate, and ethoxydiglycol is surprising and unexpected.

Table 2 provides the data pertaining to accelerated stability studies of compositions comprising naringenin dissolved in several solvents.

TABLE 2
Accelerated stability of Naringenin:
Naringenin stability at 70° C.

| | Conc (% w/w) @ Date | | | | |
|---|---|---|---|---|---|
| | Feb. 2, | Feb. 9, | Mar. 2, | 1 week | 1 month |
| | 2022 | 2022 | 2022 | stability | stability |
| Naringenin in dimethyl | 7.03 | 6.58 | 6.37 | 94% | 91% |
| isosorbide | | | | | |
| Naringenin in isopropyl | 9.28 | 8.81 | 8.63 | 95% | 93% |
| lauryol sarcosinate | | | | | |
| Naringenin in | 8.31 | 7.97 | 7.73 | 96% | 93% |
| ethoxydiglycol | | | | | |

In certain embodiments, the composition comprises naringenin in a concentration of about 5 nM to about 2500 nM. In certain embodiments, the composition comprises naringenin in a concentration of about 10 nM to about 2000 nM. In certain embodiments, the composition comprises naringenin in a concentration of about 20 nM nM to about 1000 nM. In certain embodiments, the composition comprises naringenin in a concentration of about 10 nM to about 20 nM.

In certain embodiments, in the compositions of the invention, naringenin is present in a concentration of about 0.0001% to about 5.0% w/w. In certain embodiments, in the compositions of the invention, naringenin is present in a concentration of about 0.0001% to about 1.0% w/w. In certain embodiments, in the compositions of the invention, naringenin is present in a concentration of about 0.001% to about 0.5% w/w. In certain embodiments, in the compositions of the invention, naringenin is present in a concentration of about 0.01% to about 0.5% w/w. In certain embodiments, in the compositions of the invention, naringenin is present in a concentration of about 0.1% to about 0.5% w/w. In certain embodiments, in the compositions of the invention, naringenin is present in a concentration of about 0.45% w/w.

In certain aspects, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment. In certain embodiments, the compositions of the invention also include excipients. These excipients may be selected from a group consisting of: solvent, emulsifier, preservative, antioxidant, emollient, thickening agent, penetration enhancer, surfactant, diluent, filler, carrier, and/or pH control agent. In certain embodiments, the preservative in the composition is an antimicrobial preservative or chelating agent.

The compositions of the invention may be prepared by various methods. As an example, the compositions may be prepared by dissolving, dispersing, etc naringenin as provided herein and optional excipients in a carrier (e.g., water, saline, aqueous dextrose, glycerol, glycols, ethanol or the like) or a solvent to form a solution, colloid, liposome, emulsion, complexes (including inclusion complexes), coacervate, or suspension. In certain embodiments, the compositions of the current invention can also contain auxiliary substances such as wetting agents, emulsifying agents, co-solvents, solubilizing agents, pH buffering agents, and the like (e.g., sodium acetate, sodium citrate, cyclodextrins and derivatives, sorbitan monolaurate, triethanolamine acetate, triethanolamine oleate, hydroxyethylpiperazine and the like). In certain embodiments, the compositions of the invention may further include one or more additional excipients selected from a group consisting of carriers, excipients, or diluents including, but not limited to, absorbents, anti-irritants, antibacterials, anti-acne agents, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Absorbents are substances which are added to topical products to take up water and oil-soluble dissolved or finely dispersed substances. Topical absorbents can also be used as thickeners in a wide variety of formulations including facial creams, lipsticks, shampoos and calamine lotions. In some embodiments, absorbents may include, but are not limited to, alcohol (ethyl alcohol, methanol, isopropyl alcohol, SD alcohol [especially denatured alcohol] and benzyl alcohol), alumina (aluminum oxide), aluminum chlorohydrate, aluminum hydroxide, aluminum magnesium silicate, aluminum silicate, aluminum starch octenylsuccinate, aluminum sulfate, ammonium chloride, bentonite, bismuth oxychloride, boron nitride, calcium carbonate, carnauba wax, charcoal, China clay, clay, Copernicia cerifera wax, cornstarch, fuller's earth, hydrolyzed corn starch, iron powder, kaolin, lithium magnesium sodium silicate, magnesium, magnesium carbonate, magnesium hydroxide, montmorillonite, nylon-12, rice starch, silica, silicate, silk powder, silt, sodium carbonate, sodium polyacrylates, and zeolite.

Anti-Irritants are substances which are added to topical products to reduce inflammation, especially, the inflammation resulting from the application of the product itself. The excipients included as anti-irritants that perform the function of anti-irritants or anti-inflammatories may have more than one function. For example, many antioxidants also function as anti-irritants. In some embodiments, anti-irritants and/or antioxidants may include, but are not limited to, Acacia senegal gum, acetylsalicylic acid, Achillea millefolium, adenosine, citric acid, adenosine triphosphate, Aloe barbadensis, Aloe barbadensis leaf juice extract, Aloe extract, Aloe juice, hydrogenated olive oil, hydrogenated palm glycerides, hydrolyzed silk, hydroquinone, Aloe vera, and/or alpha bisabolol.

In certain embodiments, the compositions of the invention may further include humectants. Humectants (or moisturizers) are important cosmetic ingredients that prevent loss of moisture thereby retaining the skin's natural moisture. Some humectants also can actively attract moisture. In certain embodiments, the humectants may be selected from a group consisting of sodium hyaluronate, sodium acetylated hyaluronate, hydrolyzed sodium hyaluronate, propylene glycol, hexylene glycol, and butylene glycol.

In certain embodiments, the compositions of the invention may further include slip agents. The term “slip agent” is used to describe a range of ingredients which can help other ingredients spread over the skin and penetrate into the skin. Slip agents can also have humectant (hygroscopic) properties. In some embodiments, slip agents may include, but are not limited to, amodimethicone, bis-PEG-18 methyl ether dimethyl silane, bis-phenylpropyl dimethicone, butylene glycol, cetyl dimethicone, cetyl dimethicone copolyol, cetyl PEG/PPG-10/1-dimethicone, cyclohexasiloxane, cyclomethicone, cyclopentasiloxane, cyclotetrasiloxane, decylene glycol, diisostearoyl trimethylolpropane siloxy silicate, dimethicone, dimethicone copolyol, dimethicone crosspolymer, dimethiconol, dipropylene glycol, hexylene glycol, hydrolyzed silk, isododecane, methicone, methyl trimethicone, methylsilanol mannuronate, methylsilanol PEG-7 glyceryl cocoate, Good, PEG-10 dimethicone, PEG-10 dimethicone/vinyl dimethicone crosspolymer, PEG-12 dimethicone, PEG/PPG-18/18 dimethicone, PEG/PPG-20/15 dimethicone, pentylene glycol, phenyl trimethicone, polymethylsilsesquioxane, PPG-3 benzyl ether myristate, silica dimethyl silylate, silk powder, siloxane, simethicone, sorbitol, stearyl dimethicone, stearyl methicone, triethoxycaprylylsilane, trimethylsiloxysilicate, xylitol, and zinc stearate.

In certain embodiments, the compositions of the invention may further include diluents, and/or carriers. Diluents and/or carriers may include, but are not limited to, polyethylene glycols (such as PEG200, PEG300, PEG400, PEG540, PEG600, PEG1450 or mixtures thereof) and coconut oils (such as propylene glycol dicaprate, coco-caprylate/caprate, propylene glycol dicaprylate/dicaprate, caprylic/capric triglyceride, caprylic/capric/lauric triglyceride, caprylic/capric/linoleic triglyceride, tricaprin, tricaprylin, glyceryl trioleate, neopentyl glycol dicaprylate/dicaprate, caprylic/capric/palmitic/stearic triglyceride, or mixtures thereof).

In certain embodiments, the compositions of the invention may further include emollients. Emollients are supple, waxlike, lubricating, thickening substances which are added to cosmetic/dermatological products to prevent water loss and have a softening and soothing effect on the skin. In some embodiments, emollients may include, but are not limited to, 10-hydroxydecanoic acid, acetyl glyceryl ricinoleate, acetylated castor oil, acetylated hydrogenated cottonseed glyceride, acetylated lanolin, acetylated lanolin alcohol, acetylated palm kernel glycerides, agar,% algae extract, algin, almond oil, squalane, squalene, α-glucan oligosaccharide, amodimethicone, Anacystis nidulans extract, apricot kernel oil, arachidic acid, arachidonic acid, arachidyl alcohol, arachidyl propionate, C₁₀₋₃₀ cholesterol/lanosterol esters, C¹²⁻¹⁵ alkyl benzoate, C₁₂₋₁₈ acid triglyceride, C₁₈₋₃₆ acid triglyceride, candelilla wax, Cannabis sativa L. oil, caprylic/capric triglyceride, caprylyl methicone, carrot oil, Carthamus tinctorius oil, Carya illinoensis oil, castor isostearate succinate, castor oil, Caulerpa taxifolia extract, and/or cephalin.

In certain embodiments, the compositions of the invention may further include film-forming/holding agents. Film-Forming/Holding agents are substances which are added to cosmetic/dermatological products to help leave a pliable, cohesive, and continuous covering over the skin. This film has water-binding properties and leaves a smooth feel on skin. % In some embodiments, film-forming/holding agents may include, but are not limited to, acrylate, acrylates copolymer, acrylates/C₁₀₋₃₀ alkyl acrylate crosspolymer, acrylates/dimethicone copolymer, adipic acid/neopentyl glycol/trimellitic anhydride copolymer, allyl methacrylates crosspolymer, carnauba wax, cellulose gum, Copernicia cerifera wax, dextrin, diisopropyl adipate, glyceryl polymethacrylate, hydrolyzed wheat protein, hydroxyethylcellulose, locust bean, neopentyl glycol diheptanoate, PEG-40 hydrogenated castor oil, polyacrylamide, polyacrylate-17, polyglucuronic acid, polyglyceryl methacrylate, polyisobutene, polymethyl methacrylate, polyquaternium-10, polyquaterniums, polyvinyl alcohol, polyvinylpyrrolidone, propylene carbonate, PVM/MA (polyvinyl methyl ether/maleic acid) decadiene crosspolymer, PVP [poly(vinylpyrrolidone)] copolymer, PVP/dimethylaminoethyl-methacrylate, sodium carbomer, sodium polyacrylate, styrene/acrylates copolymer, triethoxycaprylylsilane crosspolymer, VA (vinyl acetate)/crotonates, VA/crotonates copolymer, VP (vinylpyrrolidone)/eicosene copolymer, and VP/hexadecene copolymer.

In certain embodiments, the compositions of the invention may further include excipients related to the fragrance of the formulation. Fragrance ingredients are a single or a blend of volatile and/or fragrant plant oils (or synthetically derived oils) that impart aroma and odor to cosmetic/dermatological products. The level of fragrance to use varies according to the product type. In some embodiments, the composition of the formulation may contain up to about 0.01% fragrance by weight. In some embodiments, fragrances (e.g., synthetic and fragrant plant extracts) may include, but are not limited to, Acacia farnesiana extract, Aerocarpus santalinus, amyl cinnamate, amyl salicylate, amyris oil, Anethum graveolens, Angelica archangelica root oil, anisaldehyde, anise, balm mint extract, balsam peru, bay leaf oil, bergamot oil, bitter orange flower, bois de rose oil, bois oil, Boswellia carterii, butylphenyl methylpropional, Cananga extract, Cananga odorata, cardamom, cedarwood, cherry extract, Citrus aurantifolia, Citrus aurantium, Citrus aurantium extract, Citrus medica limonium, clary oil, Commiphora myrrha extract, coriander, Cucurbitea peponis, Cyclamen aldehyde, dill extract, ethyl vanillin, eugenol, farnesol, farnesyl acetate, Ferula galbaniflua, fir needle oil, floralozone, Foeniculum vulgare extract, frankencense extract, galbanun, Gardenia florida extract, grapefruit oil, guaiac wood, Guaiacum officinale, hedione, hexyl cinnamal, hyssop, Illicium vernum, Iris florentina extract, jasmine oil, Jasminium grandiflorum, jonquil extract, Laurus nobilis, lauryl lactate, lavandin oil, Lavandula angustifolia, Lavandula officinalis, lavender extract and oil, lemon, lemon balm, lemongrass oil, Levisticum officinale root extract, lime oil and extract, limonene, linalool, Litsea cubeba, mandarin orange oil or extract, marjoram, Melissa officinalis, Mentha piperita, Mentha spicata, Mentha viridis, menthol, menthone, menthoxypropanediol, and menthyl lactate.

In certain embodiments, the composition of the invention may further include preservatives. Preservatives are substances which prevent bacterial, microbial or fungal contamination of cosmetic/dermatological products thereby increasing the product's shelf life and consumer safety. Some of these agents also have stabilizing effects able to preserve the function of various active ingredients including anti-oxidants (vitamins), emulsifiers and surfactants. In some embodiments, preservatives may include, but are not limited to, 1,2-hexanediol, benzoic acid, benzothonium chloride, borax, bronopol, butylparaben, caprylyl glycol, chlorophene, chloroxylenol, chlorphenesin, decylene glycol, dehydroacetic acid, diazolidinyl urea, DMDM hydantoin, ethylhexylglycerin, ethylparaben, formaldehyde-releasing preservative, Germaben II, hoelen, hydroxyacetophenone, imidazolidinyl urea, iodopropynyl butylcarbamate, isobutylparaben, methylchloroisothiazolinone, methyldibromo glutaronitrile, Methylisothiazolinone, methylparaben, o-cymen-5-ol, phenoxyethanol, phenoxyisopropanol, phytosphingosine, polyaminopropyl biguanide, potassium sorbate, propylparaben, quaternium-15, sodium benzoate, sodium citrate, sodium dehydroacetate, sodium hexametaphosphate, sodium hydroxymethylglycinate, phenethyl alcohol, sodium lactobionate, sodium metabisulfite, sodium sulfite, sorbic acid, and Styrax benzoin.

In certain aspects of the invention, the pharmaceutical composition is an emulsion. The emulsion compositions of the invention may further include emulsifiers. Emulsifiers are substances which are added to cosmetic/dermatological products to help keep unlike ingredients (such as oil and water) from separating in an emulsion. There are 2 types of emulsifiers. Oil-in-water (o/w) emulsifiers keep oil drops packed in water, while water-in-oil (w/o) emulsifiers keep water drops packed in oil. W/O emulsifiers are used for a fatty feel (e.g. night & sun protection creams). O/W emulsifiers are used more in moisturizing products (e.g. body lotions, day creams).

In certain embodiments, the compositions of the invention may further include surfactants. Surfactants are compounds that lower surface tension or interfacial tension. Reducing the surface tension allows a liquid to spread easier while reducing the interfacial tension (interfacial meaning between two faces) between water and an oil/lipid allows them to mix. Surfactants may have a be anionic, amphoteric, non-ionic, and/or quaternary agents. Surfactants form the base of all personal cleansing products and can also have wetting, conditioning, defatting, emulsifying, & thickening effects. In some embodiments, emulsifiers, surfactants, and detergents may include, but are not limited to, ammonium laureth sulfate, ammonium lauryl sulfate, arachidyl glucoside, behenic acid, bis-PEG-18 methyl ether dimethyl silane, C₂₀₋₄₀ pareth-40, cocamidopropyl betaine, cocamidopropyl dimethylamine, cocamidopropyl hydroxysultaine, coco-glucoside, coconut oil, decyl glucoside, dicetyl phosphate, dihydrocholeth-30, disodium cocoamphodiacetate, disodium cocoyl glutamate, disodium lauraminopropionate, glyceryl behanate, hydrogenated vegetable glycerides citrate, isohexadecane, isostearamide DEA, lauramphocarboxyglycinate, laureth-23, laureth-4, laureth-7, lauryl PEG-9 polydimethylsiloxyethyl dimethicone, lauryl alcohol, lauryl glucoside, magnesium laureth sulfate, magnesium oleth sulfate, myristic acid, nonoxynols, oleic acid, oleth 10, palm kernel acid, palmitic acid, PEG-60 almond glycerides, PEG-75 shea butter glycerides, PEG 90M, PEG-10 dimethicone, PEG-10 dimethicone/vinyl dimethicone crosspolymer, PEG-10 rapeseed sterol, PEG-100 stearate, PEG-12 dimethicone, PEG-120 methyl glucose dioleate, PEG-20 methyl glucose sesquistearate, PEG-40 stearate, PEG-60 hydrogenated castor oil, PEG-7 glyceryl cocoate, PEG-8, PEG-80 sorbitan laurate, PEG/PPG-17/6 copolymer (polyethylene glycol/polypropylene glycol-17/6 copolymer), PEG/PPG-18/18 dimethicone, PEG/PPG-20/15 dimethicone, poloxamer 184, Poloxamer 407, poloxamers, polyglyceryl-3 beeswax, polyglyceryl-4 isostearate, polyglyceryl-6 isostearate, polysorbate 20, polysorbate 60, polysorbate 80, potassium cetyl phosphate, potassium hydroxide, potassium myristate, PPG-12 buteth-16, PPG-26-Buteth-26, Salvia officinalis, Saponaria officinalis extract, soapwort, sodium C₁₄₋₁₆ olefin sulfonate, sodium cetearyl sulfate, sodium cocoamphoacetate, sodium cocoate, sodium cocoyl glutamate, sodium cocoyl isethionate, sodium dilauramidoglutamide lysine, sodium hexametaphosphate, sodium hydroxide, sodium laureth sulfate, sodium laureth-13 carboxylate, sodium lauroamphoacetate, sodium lauroyl lactylate, sodium lauroyl sarcosinate, sodium lauryl glucose carboxylate, sodium lauryl sulfate, sodium methyl cocoyl taurate, sodium methyl taurate, sodium myreth sulfate, sodium palm kernelate, sodium palmate, sodium PEG-7 olive oil carboxylate, sodium trideceth sulfate, steareth-20, TEA-lauryl sulfate (triethanolamine-lauryl sulfate), and tribehenin PEG-20 esters.

In certain embodiments, the compositions of the invention may further include one or more thickeners. Thickeners are substances which are added to cosmetic/dermatological products to enhance the consistency, volume and viscosity of cosmetic products, thereby providing more stability and better performance. While some thickeners have also emulsifying or gelling properties, the majority of thickeners have the ability to retain water on the skin and act therefore as moisturizers. Thickeners can be completely natural like waxes but also synthetic or semi-synthetic. In some embodiments, thickeners may include, but are not limited to, Acacia Senegal gum, acetyl glyceryl ricinoleate, acetylated castor oil, acetylated hydrogenated cottonseed glyceride, acetylated palm kernel glycerides, acrylates/steareth-20 methacrylate copolymer, agar, Ahnfeltia concinna extract, Ahnfeltia extract, Alaria esculenta, algae, algae extract, algin, alkyloamides, Althaea rosea, Althea officinalis, alumina, aluminum hydroxide, aluminum stearate, ammonium acryloyldimethyltaurate/VP copolymer, Anacystis nidulans extract, arachidic acid, arachidonic acid, arachidyl alcohol, arachidyl propionate, arrowroot, artemia extract, Ascophyllum nodosum, ascorbyl methylsilanol pectinate, Asparagopsis armata extract, beeswax, behenic acid, behentrimonium chloride, behenyl alcohol, bis-diglyceryl polyacyladipate, bismuth oxychloride, C₁₂₋₁₅alkyl benzoate, C₁₂₋₁₈ acid triglyceride, C₁₃₋₁₄ isoparaffin, C₁₈₋₃₆ acid triglyceride, candelilla wax, caprylic/capric triglyceride, carbomer, carbopol, carnauba wax, carrageenan, Caulerpa taxifolia extract, cellulose, Cellulose gum, cephalin, Cera alba, ceresin, ceteareth-20, cetearyl alcohol, cetearyl ethylhexanoate, cetearyl glucoside, cetearyl octanoate, cetyl alcohol, cetyl dimethicone copolyol, cetyl hydroxyethylcellulose, cetyl palmitate, cetyl PEG/PPG-10/1-dimethicone, chlorella, Chondrus crispus, cocamide DEA and MEA, cocoglycerides, Codium tomentosum extract, Copernicia cerifera wax, Corallina officinalis extract, Corallina, DEA oleth-10 phosphate, diethanolamine (DEA), di-PPG-3 myristyl ether adipate, dimethicone crosspolymer, dimethicone/vinyl dimethicone crosspolymer, polyacrylate crosspolymer, and/or dimer dilinoleyl dimer dilinoleate.

In certain embodiments, the compositions of the invention may further include one or more chelators. Chelating agents are any of numerous ingredients that bind with metal ions or metallic compounds, preventing them from adhering to a surface (such as skin, hair, or clothing) or causing contamination, such as in the case of trace amounts of iron. In some embodiments, chelators may include, but are not limited to tetrasodium EDTA, sodium phytate, and/or teathydroxypropyl ethylenediamine.

In certain embodiments, the excipients included in the composition are water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and/or ethylhexylglycerin.

Tables 3-9 provide a list of excipients, along with their approximate concentrations, that may be used in the exemplary compositions of the invention.

TABLE 3
Exemplary serum composition:

| Ingredient | Concentration w/w % |
|---|---|
| Water | 30%-90% |
| Glycerin | 2-10% |
| Niacinamide | 0-10% |
| Squalane | 0-10% |
| C₁₄₋₂₂ Alcohols | 0-5% |
| Simmondsia Chinensis (Jojoba) | 0-5% |
| Seed Oil | |
| Ethoxydiglycol | 0-5% |
| Propanediol | 0-5% |
| Caprylyl Glyceryl Ether | 0-2% |
| Lauroyl Lysine | 0-2% |
| Panthenol | 0-2% |
| Butylene Glycol | 0-2% |
| Naringenin | 0.0001-5.00% |
| C12-20 Alkyl Glucoside | 0-2% |
| Polyacrylate Crosspolymer-6 | 0-2% |
| Caprylic/Capric Triglyceride | 0-2% |
| Caprylhydroxamic Acid | 0-2% |
| Butyrospermum Parkii (Shea) | 0-2% |
| Butter | |
| Cetearyl Alcohol | 0-2% |
| Palmitic Acid | 0-2% |
| Sodium Phytate | 0-2% |
| Pentylene Glycol | 0-2% |
| Allantoin | 0-2% |
| Hedychium Coronarium Root | 0-1% |
| Extract | |
| Acacia Senegal Gum | 0-1% |
| Xanthan Gum | 0-1% |
| Citric Acid | 0-1% |
| Arachidyl Alcohol | 0-1% |
| Arachidyl Glucoside | 0-1% |
| Behenyl Alcohol | 0-1% |
| Hydrogenated Olive Oil | 0-1% |
| Hydroxyethyl Acrylate/Sodium | 0-1% |
| Acryloyldimethyl Taurate | |
| Copolymer | |
| Phoenix Dacgylifera (Date) Seed | 0-0.5% |
| Sodium Acetylated Hyaluronate | 0-0.5% |
| Cetearyl Glucoside | 0-0.5% |
| Olea Europaea (Olive) Fruit Oil | 0-0.5% |
| Phenethyl Alcohol | 0-0.5% |
| Sodium Hyaluronate | 0-0.5% |
| Ascorbic Acid | 0-0.5% |
| Aloe Barbadensis Leaf Juice | 0-0.5% |
| t-Butyl Alcohol* | 0-0.1% |
| Sodium Hyaluronate Crosspolymer | 0-0.1% |
| Glucose | 0-0.1% |
| Olea Europaea (Olive) Oil | 0-0.1% |
| Unsaponifiables | |
| Sorbitan Isostearate | 0-0.1% |
| Hydrolyzed Sodium Hyaluronate | 0-0.1% |
| Myristic Acid | 0-0.1% |
| Stearic Acid | 0-0.1% |
| Ethylhexylglycerin | 0-0.1% |
| Lauric Acid | 0-0.1% |
| Dextran | 0-0.1% |
| Tripeptide-1 | 0-0.1% |
| Total | 100% |

TABLE 4
Exemplary balm composition

| Ingredient | Concentration w/w % |
|---|---|
| Squalane | 5-40% |
| Cetearyl Alcohol | 5-40% |
| Caprylic/Capric Triglyceride | 2-25% |
| C13-15 Alkane | 2-25% |
| Simmondsia Chinensis (Jojoba) | 2-30% |
| Seed Oil | |
| Helianthus Annuus (Sunflower) | 2-20% |
| Seed Wax | |
| Stearyl Behenate | 2-15% |
| C10-18-Triglycerides | 1-15% |
| Dimethyl Isosorbide | 1-15% |
| Polyhydroxystearic Acid | 0-10% |
| Silica | 0-10% |
| Glyceryl Caprylate | 0-5% |
| Naringenin | 0.0001-5.00% |
| Ceramide NP | 0-2% |
| Limnanthes Alba (Meadowfoam) | 0-2% |
| Seed Oil | |
| Glyceryl Behenate | 0-2% |
| Sodium Hyaluronate | 0-20% |
| TOTAL | 100.0000% |

TABLE 5
Exemplary cream gel composition

| Ingredient | Concentration w/w % |
|---|---|
| Water | 40-90% |
| Ethoxydiglycol | 0-10% |
| Naringenin | 0.0001-5.00% |
| Caprylic/Capric Triglyceride | 2-20% |
| Butyrospermum Parkii (Shea) Butter | 0-5% |
| Cetearyl Alcohol | 0-5% |
| Palmitic Acid | 0-5% |
| Pentylene Glycol | 0-5% |
| Propanediol | 0-2% |
| Hydroxyethyl Acrylate/Sodium | 0-2% |
| Acryloyldimethyl Taurate | |
| Copolymer | |
| Hydrogenated Olive Oil | 0-2% |
| Behenyl Alcohol | 0-2% |
| Arachidyl Alcohol | 0-2% |
| Arachidyl Glucoside | 0-2% |
| Olea Europaea (Olive) Fruit Oil | 0-2% |
| Sodium Phytate | 0-2% |
| Cetearyl Glucoside | 0-2% |
| Phenethyl Alcohol | 0-2% |
| Sorbitan Isostearate | 0-2% |
| Olea Europaea (Olive) Oil | 0-2% |
| Unsaponifiables | |
| Stearic Acid | 0-2% |
| Glucose | 0-1% |
| Myristic Acid | 0-0.1% |
| Lauric Acid | 0-0.1% |
| Citric Acid | 0-2% |
| Linoleic Acid | 0-3% |
| Lecithin | 0-3% |
| Phytosteryl Canola Glycerides | 0-2% |
| Oleic Acid | 0-2% |
| Tocopherol | 0-1% |
| Triolein | 0-1% |
| Phospholipids | 0-5% |
| Sphingolipids | 0-1% |
| Sodium Citrate | 0-1% |
| p-Anisic Acid | 0-0.1% |
| Phytosterols | 0-5% |
| C13-15 Alkane | 0-10% |
| Butylene Glycol | 0-10% |
| Glycerin | 0-10% |
| Glyceryl Stearate SE | 0-10% |
| Helianthus Annuus (Sunflower) | 0-10% |
| Seed Oil | |
| Alteromonas Ferment Extract | 0-2% |
| Sodium Acetylated Hyaluronate | 0-0.1% |
| Sodium Hyaluronate | 0-0.1% |
| Sodium Hyaluronate Crosspolymer | 0-0.1% |
| Hydrolyzed Sodium Hyaluronate | 0-0.1% |
| Ethylhexylglycerin | 0.00075% |
| Sodium Acrylates Copolymer | 0-5% |
| Centella Asiatica Extract | 0-5% |
| Cetyl Alcohol | 0-5% |
| Hydroxyacetophenone | 0-5% |
| 1,2-Hexanediol | 0-5% |
| Caprylyl Glycol | 0-5% |
| Sodium Hydroxide | 0-2% |
| Total | 100.0000% |

TABLE 6
Exemplary eye cream composition

| Ingredient | % w/w |
|---|---|
| Water | 40-90% |
| Caprylic/Capric Triglyceride | 2-20% |
| Glycerin | 1-15% |
| Silica | 1-10% |
| Dimethyl Isosorbide | 1-10% |
| Squalane | 0.5-5% |
| Diheptyl Succinate | 0.4-4% |
| Capryloyl Glycerin/Sebacic Acid | 0.5-5% |
| Copolymer | |
| Arachidyl Alcohol | 0.5-5% |
| Microcrystalline Cellulose | 0.2-5% |
| Hydroxyethyl Acrylate/Sodium | 0.2-5% |
| Acryloyldimethyl Taurate Copolymer | |
| Behenyl Alcohol | 0.2-5% |
| Hydroxyacetophenone | 0.2-5% |
| Cetearyl Olivate | 0.2-5% |
| Sortiban Olivate | 0.2-5% |
| Hydrogenated Olive Olil | 0.2-5% |
| Naringenin | 0.2-5% |
| Arachidyl Glucoside | 0.2-2% |
| Lauroyl Lysine | 0.1-2% |
| Pentylene Glycol | 0.1-2% |
| 1,2-Hexanediol | 0.1-2% |
| Caprylyl Glycol | 0.1-2% |
| Olea Europaea (Olive) Fruit Oil | 0.05-1% |
| Disodium Acetyl Glucosamine | 0.02-0.5% |
| Phosphate | |
| Sodium Glucuronate | 0.02-0.5% |
| Phospholipids | 0.02-0.5% |
| Olea Europaea (Olive) Oil | 0.01-0.2% |
| Unsaponifiables | |
| Polysorbate 60 | 0.01-0.2% |
| Sorbitan Isostearate | 0.01-0.2% |
| Magnesium Sulfate | 0.005-0.1% |
| Sodium Benzoate | 0.005-0.1% |
| Sphingolipids | 0.005-0.1% |
| Sodium Acetylated Hyaluronate | 0.005-0.1% |
| Sodium Hyaluronate | 0.005-0.1% |
| Sodium Citrate | 0.0025-0.05% |
| p-Anisic Acid | 0.0015-0.05% |
| Sodium Hyaluronate Crosspolymer | 0.0015-0.05% |
| Hydrolyzed Sodium Hyaluronate | 0.00015-0.005% |
| Acetyl Hexapeptide-8 | 0.00015-0.005% |
| Ethylhexlglycerin | 0.00015-0.005% |
| Tocopherol | 0.000015-0.0005% |
| Sum Total | 100.0000% |

TABLE 7
Exemplary sunscreen formulation

| Ingredients | % w/w |
|---|---|
| Water | 25-80% |
| Zinc Oxide | 5-40% |
| Caprylic/Capric Triglyceride | 1-20% |
| Dicaprylyl Carbonate | 1-20% |
| C13-15 Alkane | 1-20% |
| Dimethyl Isosorbide | 0.5-10% |
| Propanediol | 0.5-10% |
| Cetearyl Alcohol | 0.25-5% |
| Neopentyl Glycol Dipheptanoate | 0.25-5% |
| Polyhydroxystearic Acid | 0.25-5% |
| Polyglyceryl-3 Polyricinoleate | 0.25-5% |
| Isotearic Acid | 0.25-5% |
| Lecithin | 0.25-5% |
| Octyldodecanol | 0.25-5% |
| Coco-Glucoside | 0.25-5% |
| Trehalose | 0.25-5% |
| Potassium Cetyl Phosphate | 0.25-5% |
| Simmondsia Chinensis (jojoba) Seed Oil | 0.25-5% |
| Citric Acid | 0.1-5% |
| Isododecane | 0.1-5% |
| Cellulose | 0.1-5% |
| Caprylyl Glyceryl Ether | 0.1-5% |
| Bis-Diglyceryl Polyacyladipate-2 | 0.1-5% |
| Octyldodecanol | 0.1-5% |
| Naringenin | 0.1-5% |
| Cellulose Gum | 0.1-5% |
| Disteardimonium Hectorite | 0.1-5% |
| Iron Oxide (CI77492) | 0.2400% |
| Acacia Senegal Gum | 0.05-1% |
| Caprylhydroxamic Acid | 0.05-1% |
| Propylene Carbonate | 0.05-1% |
| Xanthan Gum | 0.05-1% |
| Glycerin | 0.05-1% |
| Sodium Phytate | 0.05-1% |
| Aloe Barbadensis Leaf Juice Powder | 0.005-0.2% |
| Glucose | 0.001-0.05% |
| Iron Oxide (CI77491) | 0.001-0.05% |
| Dunaliella Saline Extract | 0.0005-0.005% |
| Total | 100.0000% |

TABLE 8
Exemplary Cream Formulations

| Ingredient | % w/w |
|---|---|
| Water | 40-90% |
| Glycerin | 1-10% |
| Diheptyl Succinate | 1-10% |
| Caprylic/Capric Triglyceride | 1-10% |
| Dimethyl Isosorbide | 0.5-5% |
| Squalane | 0.5-5% |
| C15-19 Alkane | 0.5-5% |
| Arachidyl Alcohol | 0.1-5% |
| Pseudozyma Epicola/Sunflower Seed Oil | 0.1-5% |
| Ferment Extract Filtrate | |
| Propanediol | 0.1-5% |
| Hydroxyethyl Acrylte/Sodium | 0.1-5% |
| Acryloyldimethyl Taurate Copolymer | |
| Xylitylglucoside | 0.1-5% |
| Behenyl Alcohol | 0.1-2.5% |
| Saccharide Isomerate | 0.1-2.5% |
| Caprylyl Glyceryl Ether | 0.1-2.5% |
| Cetearyl Olivate | 0.1-2.5% |
| Sortiban Olivate | 0.1-2.5% |
| Hydrogenated Olive Oil | 0.1-2.5% |
| Anhydroxylitol | 0.1-2.5% |
| Naringenin | 0.1-5% |
| Arachidyl Glucoside | 0.1-2.5% |
| Phospholipids | 0.1-2.5% |
| Phytosterols | 0.1-2.5% |
| Linoleic Acid | 0.1-2.5% |
| Ceramide NP | 0.1-2.5% |
| Propylene Glycol | 0.05-2% |
| Xylitol | 0.05-2% |
| Caprylhydroxamic Acid | 0.05-2% |
| Olea Europaca (Olive) Fruit Oil | 0.05-2% |
| Capryloyl Glycerin/Sebacic Acid | 0.01-2% |
| Copolymer | |
| Olea Europaea (Olive) Oil | 0.01-1% |
| Unsaponifiables | |
| Glucose | 0.01-1% |
| Polysorbate 60 | 0.01-1% |
| Sorbitan Isostearate | 0.01-1% |
| Hydrogenated Lecithin | 0.01-1% |
| Glyceryl Stearate | 0.005-0.5% |
| Citric Acid | 0.005-0.5% |
| Sodium Citrate | 0.001-0.5% |
| Ceramide NS | 0.001-0.5% |
| Ceramide AG | 0.001-0.5% |
| Ceramide AP | 0.001-0.5% |
| Cholesterol | 0.001-0.5% |
| Ceramide EOP | 0.00001-0.005% |
| Total | 100.000% |

TABLE 9
Exemplary Night Mask Formulations:

| Ingredients | % w/w |
|---|---|
| Water | 40-80% |
| Gluconolactone | 2.5-25% |
| Squalane | 1-10% |
| Glycerin | 1-10% |
| Propanediol | 1-10% |
| Hemisqualane | 1-10% |
| Neopentyl Glycol Diheptanoate | 0.5-5% |
| Dimethyl Isosorbide | 0.5-5% |
| Sodium Polyacryloyldimethyl Taurate | 0.5-5% |
| Polyglyceryl-3Methylglucose Distearate | 0.5-5% |
| Isododecane | 0.5-5% |
| Hydroxyethyl Acrylate/Sodium Acryloyl | 0.1-2.5% |
| Dimethyl Taurate Copolymer | |
| Helianthus Annuus Seed Oil | 0.1-2.5% |
| Sodium Hydroxide | 0.1-2.5% |
| Hydroxyacetophenone | 0.1-2.5% |
| Naringenin | 0.1-5% |
| Inulin Lauryl Carbamate | 0.1-5% |
| Persea Gratissima (Avocado) Fruit Extract | 0.05-2.5% |
| Maltodextrin | 0.05-2.5% |
| Caprylyl Glycol | 0.05-2.5% |
| 1,2-Hexanediol | 0.05-2.5% |
| Hydrolyzed Jojoba Esters | 0.05-2.5% |
| Pentylene Glycol | 0.05-2.5% |
| Sodium Acetylated Hyaluronate | 0.005-0.25% |
| Sodium Hyaluronate | 0.0025-0.25% |
| Capsicum Annuum Fruit Extract | 0.0015-0.05% |
| Sodium Hyaluronate Crosspolymer | 0.0015-0.05% |
| Hydrolyzed Sodium Hyaluronate | 0.00015-0.005% |
| Ethylhexlglycerin | 0.0001-0.01% |
| Total | 100% |

### Treatment of Topical Conditions

Advantageously, it was discovered that naringenin impacts the genes and signaling pathways involved in several topical conditions. In one aspect, the invention provides methods of using the compositions for treating, protecting, and/or altering (e.g., improving) the condition and/or aesthetic appearance of skin, including, for example, treating, preventing, ameliorating, reducing and/or eliminating fine lines and/or wrinkles of skin and/or improving the aesthetic appearance of fine lines and/or wrinkles of skin. In certain embodiments, the compositions of the invention lead to increase in skin and/or hair resiliency. In certain embodiments, the compositions of the invention lead to increase in elasticity of the skin. In certain embodiments, the compositions of the invention prevent skin damage.

In one aspect, the invention includes a composition for topical administration comprising an effective amount of naringenin. The effective amount of naringenin is an amount of naringenin that would be effective in treating, ameliorating, or otherwise impacting the underlying condition of the skin.

In certain aspects of the invention, the compositions comprising naringenin demonstrate antioxidant activity. In certain embodiments, the compositions of the invention, the antioxidant activity of naringenin is demonstrated at concentrations of about 0.001% w/w or higher. In certain embodiments, the compositions of the invention, in aqueous medium, the antioxidant activity of naringenin is demonstrated at concentrations of about 0.005% w/w or higher. In certain embodiments, the compositions of the invention, in organic solvents, the antioxidant activity of naringenin is demonstrated at concentrations of about 0.5% w/w or higher. Advantageously, the compositions of the invention comprising naringenin, demonstrating antioxidant activity, are effective for treating and/or preventing oxidation damage. In certain embodiments, the compositions of the invention comprising naringenin, demonstrating antioxidant activity, are effective for treatment of skin and/or hair damaged by reactive oxygen species and protection from other environmental aggressors that may damage the skin of the subject. In certain other embodiments, the compositions of the invention comprising naringenin, demonstrating antioxidant activity, are effective for prevention of skin and/or hair damage by reactive oxygen species, including the damage caused by environmental aggressors.

As demonstrated from the data provided in Examples 4 and 5, compositions comprising naringenin are effective in demonstrating robust antioxidant activity in both aqueous and organic solvent systems. The IC₅₀ values for antioxidant activity calculated in aqueous solvent system was 0.0058% w/w. These results demonstrate that compositions comprising naringenin have robust antioxidant activity. In certain embodiments, the compositions comprising naringenin are useful in treatment of conditions caused by oxidation or excessive oxidation in the skin of the subject. In certain embodiments, the compositions comprising naringenin are administered for treatment and/or prevention of skin or hair damage by reaction oxygen species and protection from environmental aggressors.

In certain aspects of the invention, the compositions comprising naringenin demonstrate elastase enzyme inhibition. In certain embodiments, the compositions of the invention, the elastase enzyme inhibition activity of naringenin is demonstrated at concentrations of about 0.01% w/w or higher. In certain embodiments, the compositions of the invention, the elastase enzyme inhibition activity of naringenin is demonstrated at concentrations of about 0.02% w/w or higher. In certain embodiments, the compositions of the invention, the elastase enzyme inhibition activity of naringenin is demonstrated at concentrations of about 0.1% w/w or higher. As evident from the data provided in Example 6, compositions comprising naringenin have robust elastase enzyme inhibition activity. Indeed, the calculated IC₅₀ value for elastase inhibition by compositions comprising naringenin is 0.159% w/w.

Advantageously, the compositions comprising naringenin, demonstrating elastase enzyme inhibition activity, are effective for promoting the elasticity of the skin. In certain embodiments, the compositions of the invention comprising naringenin, demonstrating elastase enzyme inhibition, are effective in reduction of fine lines and/or wrinkles in the skin. In certain embodiments, the compositions of the invention comprising naringenin, demonstrating elastase enzyme inhibition, are effective in prevention of formation of fine lines and/or wrinkles in the skin.

In certain aspects of the invention, the compositions comprising naringenin demonstrate inhibition of glycation. In certain embodiments, the compositions of the invention, the glycation inhibition activity of naringenin is demonstrated at concentrations of about 0.001% w/w or higher. In certain embodiments, the compositions of the invention, the glycation inhibition activity of naringenin is demonstrated at concentrations of about 0.005% w/w or higher. In certain embodiments, the compositions of the invention, the glycation inhibition activity of naringenin is demonstrated at concentrations of about 0.008% w/w or higher. As evident from the data provided in Example 7, compositions comprising naringenin have robust glycation inhibition. Indeed, the calculated IC₅₀ value for glycation inhibition by compositions comprising naringenin is 0.019% w/w. Advantageously, the compositions comprising naringenin, demonstrating glycation inhibition activity, are effective for supporting and promoting skin and/or hair resiliency.

In certain aspects of the invention, the compositions comprising naringenin demonstrate anti-inflammatory activity. In certain embodiments, the compositions comprising naringenin demonstrate anti-inflammatory activity at a concentration of 0.0001% w/w or higher. In certain embodiments, the compositions comprising naringenin demonstrate anti-inflammatory activity at a concentration of 0.0045% w/w or higher. In certain embodiments, the anti-inflammatory activity of naringenin in the compositions of the invention is demonstrated by reduction in anti-inflammatory markers IL-6 and/or TNF-α in stressed keratinocytes. In certain embodiments, the anti-inflammatory activity is demonstrated in human epidermal keratinocytes. The chemokines produced by activated keratinocytes and immune cells are able to damage the skin tissue in proximity to the target of the inflammatory response. Modulation of immune response is important in maintaining skin homeostasis. If the modulation is functioning optimally, the inflammatory response of the cells would disrupt skin homeostasis. Advantageously, the anti-inflammatory activity of compositions comprising naringenin is beneficial in maintaining this skin homeostasis and promoting the health of the skin.

The data provided in Example 8 demonstrates that compositions comprising naringenin have robust anti-inflammatory activity. The data demonstrates that naringenin shows anti-inflammatory properties by reducing inflammatory markers IL-6 and TNFα to non-stressed levels in stressed keratinocytes.

In certain aspects of the invention, the composition is a composition comprising naringenin that can be applied as a mask on the skin. Accordingly, in certain embodiments, the application of the mask results in refreshing appearance of the skin.

In certain aspects, the invention provides sunscreens comprising naringenin. In certain embodiments, the sunscreens comprising naringenin provide full broad spectrum light protection and repair on inflammaging damage to sustain firmness.

In certain aspects, the invention provides fortifying eye cream comprising naringenin. In certain embodiments, the eye cream firms and hydrates the skin to reduce visible signs of inflammaging.

Genetic Expression and/or Activity Modulation

In certain aspects of the invention, a composition comprising naringenin modulates expression of one more genes that are related to a topical condition. In certain embodiments, administration of composition comprising naringenin alters one or more of a function related with the modulated genes, wherein the one or more function is selected from the group consisting of (i) altering the appearance of the skin or ameliorating a condition leading to undesired appearance of the skin, (ii) formation of fines lines or wrinkles on the skin, (iii) inflammation, (iv) barrier support maintenance, and (v) altering cell turnover or cell recycling to change the appearance of the skin. In certain embodiments, administration of composition comprising naringenin alters expression and/or function of one or more genes are selected from a group consisting of: LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, MIR205, CCL11, CSF2, IL23A, IL11, BMP6, IL13RA2, C2CD4A, NR4A2, C2CD4B, FGF2, FABP4, GDF15, THBS1, HSPA1A, PTGS2, and/or DKK1.

In certain embodiments, administration of a composition comprising naringenin leads to up-regulation of LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, and/or MIR205. In certain embodiments, administration of a composition comprising naringenin leads to down-regulation of CCL11, CSF2, IL23A, IL11, BMP6, IL13RA2, C2CD4A, NR4A2, C2CD4B, FGF2, FARP4, GDF15, THBS1, HSPA1A, PTGS2, and/or DKK1. Tables 10 and 11 provide data for upregulation and downregulation respectively of certain genes after administration of compositions comprising 1% naringenin.

TABLE 10
Upregulation of expression and/or activity of genes upon
administration of compositions comprising 1% naringenin

| | Fold- | | |
|---|---|---|---|
| | increase 1% | | |
| Gene | naringenin | Gene name | Interpretation of function |
| LCE5A | 33 | Late cornified | Improved formation of outer skin |
| | | envelope protein 5A | protective barrier |
| KRT77 | 12 | Keratin 77 | Improved keratin production |
| ALOXE3 | 12 | Arachidonate | Improved formation of epidermis lipids, |
| | | lipoxygenase 3 | prevention of water loss through skin |
| KRT1 | 7 | Keratin 1 | Improved skin strength and resiliency |
| HPGD | 10 | Hydroxyprostaglandin | Anti-inflammatory function, degrades |
| | | dehydrogenase | inflammatory signaling compounds |
| MUCL1 | 11 | Mucin like protein 1 | Improved cell proliferation (wound |
| | | | healing) |
| CASP14 | 11 | Cysteine-aspartic | Improved skin barrier formation |
| | | acid protease 14 | |
| WFDC12 | 11 | Whey acidic protein | Likely a response to increase keratin |
| | | WFDC12 | production |
| KRT10 | 6 | Keratin 10 | Improved keratin production |
| DNASE1L2 | 9 | Deoxyribonuclease-1- | Improved recycling of dying cells |
| | | like 2 | |
| IL37 | 11 | Interleukin 37 | Anti-inflammatory function, promotes |
| | | | production of anti-inflammatory |
| | | | cytokines |
| SERPINB12 | 13 | Serine protease | Improved protection of epithelial cells |
| | | inhibitor | |
| HAL | 8 | Histidine ammonia- | More urocanic acid production - better |
| | | lyase | UV protection |
| UPK1A | 10 | Uroplakin 1A | enhanced cell proliferation |
| CALML5 | 7 | Calmodulin like 5 | associated with improved cell growth |
| LCE2B | 9 | Late cornified | Improved formation of outer skin |
| | | envelope 2B | protective barrier |
| MIR205 | 6 | MicroRNA 205 | Improves clearance of damaged cells |

TABLE 11
Downregulation of expression and/or activity of genes upon
administration of compositions comprising 1% naringenin

| | Fold- | | |
|---|---|---|---|
| | increase 1% | | |
| Gene | naringenin | Gene name | Interpretation of function |
| CCL11 | −20 | Eosinophil chemotactic | Anti-aging, reduced inflammation |
| | | protein | |
| CSF2 | −18 | Granulocyte-macrophage | Reduced inflammation, anti- |
| | | colony-stimulating factor | arthritis |
| IL23A | −18 | Interleukin-23A | Reduced inflammation |
| IL11 | −14 | Interleukin 11 | Reduced inflammation, decreased |
| | | | cancer risk |
| BMP6 | −11 | Bone morphogenic | Decreased scarring for wound |
| | | protein | healing |
| IL13RA2 | −10 | Interleukin 13 receptor | Improved recovery after |
| | | subunit alpha 2 | inflammatory stressors |
| C2CD4A | −10 | C2 calcium dependent | Reduced inflammation |
| | | domain containing 4A | |
| NR4A2 | −9 | Nuclear receptor | Reduced inflammation |
| | | subfamily 4 group A | |
| | | member 2 | |
| C2CD4B | −8 | C2 calcium dependent | Reduced inflammation |
| | | domain containing 4B | |
| FGF2 | −7 | Fibroblast growth factor | Decreased cancer risk |
| | | 2 | |
| FABP4 | −7 | Fatty acid binding | Reduced metabolic inflammation |
| | | protein 4 | from fat tissue signaling |
| GDF15 | −6 | Growth differentiation | Reduced inflammation |
| | | factor 15 | |
| THBS1 | −6 | Thrombospondin 1 | Decreased cancer risk |
| HSPA1A | −6 | Heat shock protein | Improved clearance of aged |
| | | family A (Hsp70) | cells, senolytic effect |
| | | member 1A | |
| PTGS2 | −6 | Prostaglandin- | Decreased production of |
| | | endoperoxide synthase 2 | inflammatory signaling molecules |
| DKK1 | −5 | Dickkopf WNT signaling | Reduced inflammation, decreased |
| | | pathway inhibitor 1 | aging-related bone loss |

In certain aspects of the invention, administration of a composition comprising naringenin enhances anti-inflammatory activity. In certain embodiments, administration of a composition comprising naringenin enhances anti-inflammatory activity because it leads to downregulation of CSF2, IL23A, IL11, IL13RA2, C2CD4A, NR4A2, FABP4, GDF15, and/or PTGS2. In certain embodiments, administration of a composition comprising naringenin enhances anti-inflammatory activity because it leads to upregulation of HPGD.

Granulocyte-macrophage colony-stimulating factor (CSF2) plays a role in inflammation and arthritis. In certain embodiments of the invention, downregulation of CSF2 leads to an anti-inflammatory activity and/or anti-arthritic activity. In certain embodiments, the administration of composition comprising naringenin results in at least about 10 to about 20-fold downregulation of expression and/or activity of CSF2. In certain embodiments, the administration of composition comprising naringenin results in at least about fifteen (15) fold downregulation of expression and/or activity of CSF2. The data provided in Table 10 demonstrates that administration of a composition comprising 1% naringenin results in about eighteen (18) fold downregulation in expression and/or activity of CSF2.

Interleukin 23A (1L23A) plays a role in the inflammatory pathway. In certain embodiments of the invention, downregulation of IL23A leads to an anti-inflammatory activity. In certain embodiments, the administration of composition comprising naringenin results in at least about 10 to about 20-fold downregulation of expression and/or activity of 1L23A. In certain embodiments, the administration of composition comprising naringenin results in at least about fifteen (15) fold downregulation of expression and/or activity of IL23A. The data provided in Table 10 demonstrates that administration of a composition comprising 1% naringenin results in about eighteen (18) fold downregulation in expression and/or activity of IL23A.

Interleukin 11 (IL11) plays a role in the inflammatory pathway and in certain pathways that may be involved in cancer. In certain embodiments of the invention, downregulation of IL11 leads to an anti-inflammatory activity and/or anticancer activity. In certain embodiments, the administration of composition comprising naringenin results in at least about 10 to about 20-fold downregulation of expression and/or activity of IL11. In certain embodiments, the administration of composition comprising naringenin results in at least about ten (10) fold downregulation of expression and/or activity of IL11. The data provided in Table 10 demonstrates that administration of a composition comprising 1% naringenin results in about fourteen (14) fold downregulation in expression and/or activity of IL11.

Interleukin 13 receptor subunit alpha 2 (IL13RA2) plays a role in recovery after stressor caused inflammatory response. In certain embodiments, downregulation of IL13RA2 leads to improved recovery after inflammation caused by stressors. In certain embodiments, the administration of composition comprising naringenin results in at least about 5 to about 15-fold downregulation of expression and/or activity of IL13RA2. In certain embodiments, the administration of composition comprising naringenin results in at least about ten (10) fold downregulation of expression and/or activity of IL13RA2. The data provided in Table 10 demonstrates that administration of a composition comprising 1% naringenin results in about ten (10) fold downregulation in expression and/or activity of IL13RA2.

C2 calcium dependent domain containing 4B (C2CD4B) plays a role in the inflammatory response. In certain embodiments, downregulation of C2CD4B results in reduction in inflammation. In certain embodiments, the administration of composition comprising naringenin results in at least about 5 to about 15-fold downregulation of expression and/or activity of C2CD4B. In certain embodiments, the administration of composition comprising naringenin results in at least about ten (10) fold downregulation of expression and/or activity of C2CD4B. The data provided in Table 10 demonstrates that administration of a composition comprising 1% naringenin results in about eight (8) fold downregulation in expression and/or activity of C2CD4B.

Nuclear receptor subfamily 4 group A member 2 (NR4A2) plays a role in the inflammatory response. In certain embodiments, downregulation of NR4A2 results in reduction in inflammation. In certain embodiments, the administration of composition comprising naringenin results in at least about 5 to about 15-fold downregulation of expression and/or activity of NR4A2. In certain embodiments, the administration of composition comprising naringenin results in at least about ten (10) fold downregulation of expression and/or activity of NR4A2. The data provided in Table 10 demonstrates that administration of a composition comprising 1% naringenin results in about nine (9) fold downregulation in expression and/or activity of NR4A2.

Fatty acid binding protein 4 (FABP4) plays a role in metabolic inflammation from the fat tissue. FABP4 acts at the interface of metabolic and inflammatory pathways in adipocytes and macrophages and plays important roles in the development of insulin resistance, diabetes mellitus, and atherosclerosis. In certain embodiments, downregulation of FABP4 leads to reduced metabolic inflammation from the fat tissue signaling. In certain embodiments, the administration of composition comprising naringenin results in at least about 2 to about 10-fold downregulation of expression and/or activity of FABP4. In certain embodiments, the administration of composition comprising naringenin results in at least about five (5) fold downregulation of expression and/or activity of FABP4. The data provided in Table 10 demonstrates that administration of a composition comprising 1% naringenin results in about seven (7) fold downregulation in expression and/or activity of FABP4.

Growth differentiation factor 15 (GDF15) plays a role in the inflammatory response. In certain embodiments, downregulation of GDF15 results in reduction in inflammation. In certain embodiments, the administration of composition comprising naringenin results in at least about 2 to about 10-fold downregulation of expression and/or activity of GDF15. In certain embodiments, the administration of composition comprising naringenin results in at least about five (5) fold downregulation of expression and/or activity of GDF15. The data provided in Table 10 demonstrates that administration of a composition comprising 1% naringenin results in about six (6) fold downregulation in expression and/or activity of GDF15.

Prostaglandin-endoperoxide synthase 2 (PTGS2) plays a role in the production of signaling molecules that are involved in the inflammatory response. For example, PTGS2 is involved in the conversion of arachidonic acid to prostaglandin H2, an important precursor of prostacyclin, which is expressed in inflammation. In certain embodiments, downregulation of PTGS2 leads to reduction in production of signaling molecules leading to inflammation. Thus, the downregulation of PTGS2 leads to reduced inflammation. In certain embodiments, the administration of composition comprising naringenin results in at least about 2 to about 10-fold downregulation of expression and/or activity of PTGS2. In certain embodiments, the administration of composition comprising naringenin results in at least about five (5) fold downregulation of expression and/or activity of PTGS2. The data provided in Table 10 demonstrates that administration of a composition comprising 1% naringenin results in about six (6) fold downregulation in expression and/or activity of PTGS2.

Hydroxyprostaglandin dehydrogenase (HPGD) has an anti-inflammatory function. In particular, it is involved in degradation of molecules that lead to inflammatory signaling. For example, HPGD is involved in degradation of prostaglandin, which is involved in inflammatory signaling pathway. In certain embodiments, upregulation of expression and/or activity of HPGD leads to an anti-inflammatory effect. In certain embodiments, upregulation of HPGD leads to increase in degradation of signaling molecules that are involved in inflammation. Thus, the upregulation of HPGD leads to reduced inflammation. In certain embodiments, the administration of composition comprising naringenin results in at least about 5 to about 20-fold upregulation of expression and/or activity of HPGD. In certain embodiments, the administration of composition comprising naringenin results in at least about ten (10) fold upregulation of expression and/or activity of HPGD. The data provided in Table 6 demonstrates that administration of a composition comprising 1% naringenin results in about ten (10) fold upregulation in expression and/or activity of HPGD.

In certain aspects of the invention, administration of a composition comprising naringenin enhances the skin barrier strength and/or maintenance of skin barrier lipids. In certain embodiments, administration of a composition comprising naringenin enhances the skin barrier strength and/or maintenance of skin barrier lipids because it leads to upregulation of LCE5A, KRT77, ALOXE3, KRT1, CASP14, KRT10, and/or LCE2B.

Late cornified envelope protein 5A (LCE5A) is involved in formation of outer skin protective barrier. LCE5A may also be involved in keratinization. In certain embodiments, upregulation of expression and/or activity of LCE5A results in improved formation of the outer skin protective barrier. Thus, administration of compositions comprising naringenin, which leads to upregulation of LCE5A, results in increased skin barrier strength. In certain embodiments, the administration of composition comprising naringenin results in at least about 20 to about 50-fold upregulation of expression and/or activity of LCE5A. In certain embodiments, the administration of composition comprising naringenin results in at least about twenty-five (25) fold upregulation of expression and/or activity of LCE5A. The data provided in Table 10 demonstrates that administration of a composition comprising 1% naringenin results in about thirty-three (33) fold upregulation in expression and/or activity of LCE5A.

Kertain 77 (KRT77) is involved in production of keratin. In particular, KRT77 encodes an epithelial keratin that is expressed in the skin and eccrine sweat glands. KRT77 encodes keratin 77, a member of the type II keratin family of intermediate filament proteins. Keratin 77 is also known as KRT1B, Type II cytoskeletal 1b, Type IT keratin Kb39, and cytokeratin 1B. In certain embodiments, upregulation of expression and/or activity of KRT77, results in enhanced keratin production. The increased keratin production contributes to increase in the skin barrier strength. In certain embodiments, the administration of composition comprising naringenin results in at least about 5 to about 20-fold upregulation of expression and/or activity of KRT77. In certain embodiments, the administration of composition comprising naringenin results in at least about ten (10) fold upregulation of expression and/or activity of KRT77. The data provided in Table 10 demonstrates that administration of a composition comprising 1% naringenin results in about twelve (12) fold upregulation in expression and/or activity of KRT77.

Arachidonate lipoxygenase 3 (ALOXE3) is involved in formation of epidermis lipids. In particular, ALOXE3 helps add an oxygen molecule to certain fatty acids to produce substances called fatty acid hydroperoxides. The enzyme encoded by ALOXE3 is a hydroperoxide isomerase that synthesizes a unique type of epoxy alcohol (8R-hydroxy-11R,12R-epoxyeicosa-5Z,9E,14Z-trienoic acid) from 12R-hydroperoxyeicosatetraenoic acid (12R-HPETE). This epoxy alcohol can activate the nuclear receptor peroxisome proliferator-activated receptor alpha (PPARalpha), which is implicated in epidermal differentiation. Loss of function of the enzyme encoded by this gene results in ichthyosis, implicating the function of this gene in the differentiation of human skin. ALOXE3 also assists in prevention of water loss through skin. In certain embodiments, the administration of composition comprising naringenin results in upregulation of ALOXE3, which has a role in enhancing the barrier support in the skin. In certain embodiments, the administration of composition comprising naringenin results in at least about 5 to about 20-fold upregulation of expression and/or activity of ALOXE3. In certain embodiments, the administration of composition comprising naringenin results in at least about ten (10) fold upregulation of expression and/or activity of ALOXE3. The data provided in Table 10 demonstrates that administration of a composition comprising 1% naringenin results in about twelve (12) fold upregulation in expression and/or activity of ALOXE3.

Keratin 1 (KRT1) plays a role in maintaining the skin strength and resiliency. Keratin 1 is produced in keratinocytes in the outer layer of the skin (the epidermis), including the skin on the palms of the hands and soles of the feet. The keratin 1 protein partners with another keratin protein, either keratin 9 or keratin 10, to form molecules called keratin intermediate filaments. These filaments assemble into strong networks that provide strength and resiliency to the skin and protect it from being damaged by friction and other everyday physical stresses. In certain embodiments, the administration of composition comprising naringenin results in upregulation of KRT1, which has a role in enhancing the strength and resiliency of the skin. In certain embodiments, the administration of composition comprising naringenin results in at least about 2 to about 15-fold upregulation of expression and/or activity of KRT1. In certain embodiments, the administration of composition comprising naringenin results in at least about five (5) fold upregulation of expression and/or activity of KRT1. The data provided in Table 10 demonstrates that administration of a composition comprising 1% naringenin results in about seven (7) fold upregulation in expression and/or activity of KRT1.

Cysteine-aspartic acid protease 14 (CASP14) plays a role in keratinocyte terminal differentiation, which is important for the formation of the skin barrier. In certain embodiments, administration of compositions comprising naringenin leads to upregulation of CASP14. In certain embodiments, this upregulation of CASP14 leads to the improved skin barrier formation. In certain embodiments, the administration of composition comprising naringenin results in at least about 5 to about 20-fold upregulation of expression and/or activity of CASP14. In certain embodiments, the administration of composition comprising naringenin results in at least about ten (10) fold upregulation of expression and/or activity of CASP14. The data provided in Table 10 demonstrates that administration of a composition comprising 1% naringenin results in about eleven (11) fold upregulation in expression and/or activity of CASP14.

Keratin 10 (KRT10) is also involved in keratin production. Keratin is involved in increasing the barrier strength and resilience of the skin. Keratin 10 is produced in keratinocytes in the outer layer of the skin (the epidermis). In the fluid-filled space inside these cells (the cytoplasm), the keratin 10 protein partners with a similar protein, keratin 1 (produced from the KRT1 gene), to form molecules called keratin intermediate filaments. These filaments assemble into strong networks that provide strength and resiliency to the skin and protect it from being damaged by friction and other everyday physical stresses. In certain embodiments, the administration of composition comprising naringenin results in upregulation of KRT10, which has a role in enhancing the strength and resiliency of the skin. In certain embodiments, the administration of composition comprising naringenin results in at least about 2 to about 15-fold upregulation of expression and/or activity of KRT10. In certain embodiments, the administration of composition comprising naringenin results in at least about five (5) fold upregulation of expression and/or activity of KRT10. The data provided in Table 10 demonstrates that administration of a composition comprising 1% naringenin results in about six (6) fold upregulation in expression and/or activity of KRT10.

Late cornified envelope 2B (LCE2B) plays a role in the formation of precursors of the cornified envelope of the stratum corneum. It also leads to improved formation of outer skin protective barrier. In certain embodiments, the administration of composition comprising naringenin results in upregulation of LCE2B, which has a role in enhancing the outer skin protective barrier. In certain embodiments, the administration of composition comprising naringenin results in at least about 5 to about 20-fold upregulation of expression and/or activity of LCE2B. In certain embodiments, the administration of composition comprising naringenin results in at least about five (5) fold upregulation of expression and/or activity of LCE2B. The data provided in Table 10 demonstrates that administration of a composition comprising 1% naringenin results in about nine (9) fold upregulation in expression and/or activity of LCE2B.

In certain aspects of the invention, administration of a composition comprising naringenin leads to enhancement of cell turnover and/or cell recycling. Beneficially, this enhancement of cell turnover and/or cell recycling leads to an antiaging effect. In certain embodiments, the antiaging effect may be demonstrated by reduction and/or prevention of formation of fine lines and/or wrinkles in the skin. In certain embodiments, administration of a composition comprising naringenin enhances the cell turnover and/or cell recycling in the skin because the administration of the composition leads to upregulation of DNASE1L2, SERPINB12, UPK1A, CALML5 and/or MIR205. In certain embodiments, administration of a composition comprising naringenin enhances the cell turnover and/or cell recycling in the skin because the administration of the composition leads to downregulation of BMP6 and/or HSPA1A.

Deoxyribonuclease-1-like 2 (DNASE1L2) plays a role in recycling of dying cells. Hair, nails, and the cornified layer of the epidermis, i.e. the stratum corneum, consist of cornified keratinocytes, also known as corneocytes. The process of cornification involves the cross-linking of structural proteins and leads to the high mechanical stability of corneocytes which, along with the formation of stable desmosomal links between cells, is essential for the resistance of epidermal structures to stress from the environment DNASE1L2 is a keratinocyte-specific DNase. DNASE1L2 breakdown of the nucleus in skin appendages and distinct epithelia whereas cornification of the interfollicular epidermis. Thus, DNASE1L2 plays a critical role in the recycling of cell recycling and turnover in the skin. In certain embodiments, the administration of a composition comprising naringenin leads to an upregulation of DNASE1L2, which plays a role in improved recycling of dying cells and increased cell turnover/recycling. In certain embodiments, the administration of composition comprising naringenin results in at least about 5 to about 20-fold upregulation of expression and/or activity of DNASE1L2. In certain embodiments, the administration of composition comprising naringenin results in at least about five (5) fold upregulation of expression and/or activity of DNASE1L2. The data provided in Table 10 demonstrates that administration of a composition comprising 1% naringenin results in about nine (9) fold upregulation in expression and/or activity of DNASE1L2.

Serine protease inhibitor (SERPINB12) plays a role in protection of epithelial cells. The intracellular serine protease inhibitors (serpins) are an important family of proteins that protect cells form proteinase-mediated injury. SERPINB12 is broadly expressed and is likely to protect cells, including epithelial cells, from both endogenous and exogenous peptidases. In certain embodiments, compositions comprising naringenin leads to upregulation of SERPINB12, which leads to increase in protection of epithelial cells. In certain embodiments, the administration of composition comprising naringenin results in at least about 5 to about 20-fold upregulation of expression and/or activity of SERPINB12. In certain embodiments, the administration of composition comprising naringenin results in at least about ten (10) fold upregulation of expression and/or activity of SERPINB12. The data provided in Table 10 demonstrates that administration of a composition comprising 1% naringenin results in about thirteen (13) fold upregulation in expression and/or activity of SERPINB12.

Uroplakin 1A (UPK1A) is involved in cell proliferation. The proteins in this family mediate signal transduction events that play a role in the regulation of cell development, activation, growth and motility. This encoded protein is found in the asymmetrical unit membrane (AUM) where it can complex with other transmembrane 4 superfamily proteins. UP1A may play a role in normal bladder epithelial physiology, possibly in regulating membrane permeability of superficial umbrella cells or in stabilizing the apical membrane through AUM/cytoskeletal interactions. In certain embodiments, compositions comprising naringenin leads to upregulation of UPK1A, which leads to increase cell proliferation, including the cells in the skin and the underlying tissues. In certain embodiments, this demonstrates an anti-aging effect. In certain embodiments, the administration of composition comprising naringenin results in at least about 5 to about 20-fold upregulation of expression and/or activity of UPK1A. In certain embodiments, the administration of composition comprising naringenin results in at least about ten (10) fold upregulation of expression and/or activity of UPK1A. The data provided in Table 10 demonstrates that administration of a composition comprising 1% naringenin results in about ten (10) fold upregulation in expression and/or activity of UPK1A.

Calmodulin like 5 (CALML5) plays a role in cell growth. The enzyme encoded by CALML5 is a novel calcium binding protein expressed in the epidermis and related to the calmodulin family of calcium binding proteins. It plays a key role in cell growth and proliferation, including playing a key role in terminal differentiation of keratinocytes. In certain embodiments, administration of compositions comprising naringenin leads to upregulation of CALML5, which leads to increase cell proliferation, including the cells in the skin and the underlying tissues. In certain embodiments, this demonstrates an anti-aging effect. In certain embodiments, the administration of composition comprising naringenin results in at least about 2 to about 10-fold upregulation of expression and/or activity of CALML5. In certain embodiments, the administration of composition comprising naringenin results in at least about five (5) fold upregulation of expression and/or activity of CALML5. The data provided in Table 10 demonstrates that administration of a composition comprising 1% naringenin results in about seven (7) fold upregulation in expression and/or activity of CALML5.

MicroRNA 205 (MIR205) plays a role in clearance of damaged cells. In certain embodiments, administration of compositions comprising naringenin leads to upregulation of MIR205, which leads to increase clearance of damaged cells. This increase in clearance of damaged cells leads to enhancement in cell turnover and/or recycling, which results in an improvement in anti-aging effect. Thus, in certain embodiments, this demonstrates an anti-aging effect. In certain embodiments, the administration of composition comprising naringenin results in at least about 2 to about 10-fold upregulation of expression and/or activity of MIR205. In certain embodiments, the administration of composition comprising naringenin results in at least about five (5) fold upregulation of expression and/or activity of MIR205. The data provided in Table 10 demonstrates that administration of a composition comprising 1% naringenin results in about six (6) fold upregulation in expression and/or activity of MIR205.

Bone morphogenetic protein 6 (BMP6) plays a role in wound healing. The protein encoded by this gene is a member of the TGFβ superfamily. Bone morphogenetic proteins are known for their ability to induce the growth of bone and cartilage. BMP6 may induce all osteogenic markers in mesenchymal stem cells. In certain embodiments, the administration of compositions comprising naringenin leads to downregulation of expression and/or activity of BMP6. This results in decreased scarring in wound healing. In certain embodiments, this may also lead to anti-aging effect for the skin. In certain embodiments, the administration of composition comprising naringenin results in at least about 2 to about 20-fold downregulation of expression and/or activity of BMP6. In certain embodiments, the administration of composition comprising naringenin results in at least about ten (10) fold downregulation of expression and/or activity of BMP6. The data provided in Table 11 demonstrates that administration of a composition comprising 1% naringenin results in about eleven (11) fold downregulation in expression and/or activity of BMP6.

Heat shock protein family A (Hsp70) member 1A (HSPA1A) plays a role in clearing of aged cells and/or senolytic effect. In certain embodiments, the administration of compositions comprising naringenin leads to downregulation of expression and/or activity of HSPA1A. This results in increased clearance of aged cells. In certain embodiments, this may also lead to anti-aging effect for the skin. In certain embodiments, the administration of composition comprising naringenin results in at least about 2 to about 10-fold downregulation of expression and/or activity of HSPA1A. In certain embodiments, the administration of composition comprising naringenin results in at least about five (5) fold downregulation of expression and/or activity of HSPA1A. The data provided in Table 11 demonstrates that administration of a composition comprising 1% naringenin results in about six (6) fold downregulation in expression and/or activity of HSPA1A.

In certain embodiments, administration of a composition comprising naringenin to a subject leads to the treatment or prevention of a topical condition of the subject through the modulation of levels of expression or activity of one or genes that may be involved in the topical condition. In certain embodiments, administration of a composition comprising naringenin alters one or more of a function related with the modulated genes, wherein the one or more function is selected from the group consisting of: (i) cell turnover, (ii) inflammation, (iii) barrier support, and (iv) any combination thereof.

### Treatment of Topical Conditions

The current invention recognizes that the compositions comprising naringenin is beneficial for treating, protecting and/or improving the condition and/or aesthetic appearance of skin. For example, the compositions of the invention may be effective for altering the aesthetic appearance of skin associated with or affected by, or for treating or preventing, fine lines and/or wrinkles of skin caused by, for example, cellular senescence, environmental damage or dermatoheliosis. The invention also provides methods for stimulating skin cell renewal, increasing cell or tissue regeneration, promoting fibroblast proliferation and synthesizing elastin, collagen, proteoglycans, and/or new connective tissue, thereby reducing or improving the appearance of wrinkles, restoring elasticity, resiliency, and/or suppleness to the skin.

The compositions of the invention provided herein can be useful for improving the aesthetic appearance of skin. Such improvements can include, but are not limited to, all outward visibly and tactilely perceptible manifestations as well as any other macro or micro effects due to skin aging and damage Such manifestations and effects can be induced or caused by intrinsic factors and/or extrinsic factors, e.g., chronological aging, environmental damage, climate, sun (UV) exposure, smoking, drugs, alcohol consumption, jetlag, night work, changes in circadian rhythm, pregnancy, menopause, genetic factors, nutritional factors and/or deficiencies, dehydration, stress, allergies (e.g., to plants, animals, medications, and other substances), exposure to industrial and/or household chemicals, indoor heating and cooling, various disorders and diseases such as arteriosclerosis, diabetes, heart disease, liver disease, and obesity, thinning of the outer layer of skin, decreases in the number of pigment-containing cells, increases in the size of pigment-containing cells, changes in the connective tissue, and reduction in the strength and elasticity of the skin.

The aesthetic appearance of skin can be improved, for example, by improving the appearance of skin associated with or affected by one or more of wrinkles, dry skin, sensitive skin; wrinkling and sagging; acne; vitiligo (skin condition in which there is a loss of brown color (pigment) from areas of skin); fine lines, wizened skin, thinning of the dermis, the degradation of collagen fibers, flaccid skin, thinned skin, and skin exposed to ultraviolet radiation. In some embodiments, the compositions of the invention can improve the aesthetic appearance of skin by decreasing the appearance of fine lines in the skin; creating a more youthful appearance of the skin; decreasing bags and/or rings around the eyes; increasing or restoring the elasticity, resiliency, and/or suppleness of the skin; increasing the apparent thickness, elasticity, flexibility, radiance, glow, and plumpness of the skin; improving the fineness of the skin texture; improving the appearance of wrinkles, lined, dry, flaky, aged, and/or photodamaged skin; treating or preventing photodamaged skin; reducing the signs of skin aging; reducing the appearance of hyperpigmentation; treating or preventing hyperpigmentation; treating or preventing pigment deposition in the skin (e.g., that caused by UV exposure); reducing the appearance of skin discolorations; and whitening, lightening, and/or bleaching the skin.

In certain embodiments, the compositions may be used for care, treatment, cleansing, and/or protective products for facial or body skin; anti-wrinkle or anti-aging compositions; skin firming compositions; skin lightening compositions; compositions for irritated skin; sunscreen compositions, artificial tanning (self-tanning) compositions or after-sun care compositions; scalp care compositions; shaving preparation compositions; depilatory compositions; or make-up products for the skin of the face or body.

In certain embodiments, the compositions comprising naringenin are administered for altering the aesthetic appearance of skin associated with or affected by, or treating or preventing, a skin condition/disorder (e.g., a skin condition/disorder accompanied with a loss of skin elasticity).

In certain embodiments, the compositions comprising naringenin are administered for improving the barrier function and viability of the skin.

In certain embodiments, the compositions comprising naringenin are administered for altering the aesthetic appearance of skin associated with or affected by wrinkling, sagging, and/or a loss of skin elasticity.

In certain embodiments, the compositions comprising naringenin are administered for altering the aesthetic appearance of skin associated with or affected by deteriorations in skin viscoelasticity.

In certain embodiments, the compositions comprising naringenin are administered for altering the aesthetic appearance of skin associated with or affected by one or more of wrinkles and/or fine lines, wizened skin, a lack of elasticity and/or of tonus of the skin, thinning of the dermis, degradation of collagen fibers, flaccid skin, thinned skin, and the internal degradation of the skin following exposure to ultraviolet radiation.

In certain embodiments, the compositions comprising naringenin are administered for decreasing the appearance of fine lines and/or wrinkles in the skin.

In certain embodiments, the compositions comprising naringenin are administered for decreasing the appearance of bags and/or rings around the eyes.

In certain embodiments, the compositions comprising naringenin are administered for reducing the appearance of hyperpigmentation.

In certain embodiments, the compositions comprising naringenin are administered for improving or increasing one or more of the thickness, elasticity, flexibility, radiance, glow, and plumpness of the skin.

In certain embodiments, the compositions comprising naringenin are administered for improving the fineness of skin texture.

In certain embodiments, the compositions comprising naringenin are administered for improving the appearance of wrinkled, lined, dry, flaky, aged or photodamaged skin.

In certain embodiments, the compositions comprising naringenin are administered for altering the aesthetic appearance of skin associated with or affected by skin discolorations.

### Methods of Administration:

In one aspect, the invention provides methods of administration of the compositions comprising naringenin. In certain embodiments, the invention the compositions of the invention are designed for topical administration.

In certain embodiments, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment.

In certain other embodiments, the compositions comprising naringenin may be prepared and used in the form of an aerosol spray, cream, emulsion, solid, liquid, dispersion, foam, oil, gel, lotion, mousse, ointment, powder, patch, pomade, solution, pump spray, stick, towelette, soap, or other forms commonly employed in the art of topical administration and/or cosmetic and skin care formulation. The compositions can be in an emulsion form. In addition, compounds provided herein used in the compositions provided herein can be used in color cosmetic compositions such as foundation makeups, blushes, eyeshadows, mascaras, concealers, eyeliners, lip colors, nail colors, and so on. Other cosmetic compositions can include perfumes, lipsticks, fingernail and toenail polish, eye and facial makeup, towelettes, deodorants, hand sanitizer, baby products, bath oils, bubble baths, and butters.

In certain embodiments, the compositions comprising naringenin are administered over an area of about 1.0 μg/cm² to about 100 μg/cm².

In certain embodiments, the compositions comprising naringenin are administered for a duration till the desired impact in amelioration or prevention of a topical condition is realized. In certain embodiments, the composition comprising naringenin comprises an effective amount of naringenin. The effective amount of naringenin is an amount of naringenin that results in the desired result in the condition of the skin. In certain embodiments, the composition comprising naringenin is administered once a day. In certain embodiments, the composition comprising naringenin is administered twice a day. In certain embodiments, the composition comprising naringenin is administered thrice a day. In certain embodiments, the composition comprising naringenin is administered once every two days. In certain embodiments, the composition comprising naringenin is administered once every three days.

### EXAMPLES

### Example 1: Exemplary Composition Comprising Naringenin

Table 12 provides an exemplary formulation of naringenin:

TABLE 12
Serum comprising naringenin:

| Ingredient | Concentration w/w % |
|---|---|
| Water | 72.22742% |
| Glycerin | 7.2710% |
| Niacinamide | 5.0000% |
| Squalane | 5.0000% |
| C14-22 Alcohols | 1.5900% |
| Simmondsia Chinensis (Jojoba) | 1.5000% |
| Seed Oil | |
| Ethoxydiglycol | 1.44675% |
| Propanediol | 1.25715% |
| Caprylyl Glyceryl Ether | 0.5000% |
| Lauroyl Lysine | 0.5000% |
| Panthenol | 0.5000% |
| Butylene Glycol | 0.4999% |
| Naringenin | 0.4501% |
| C12-20 Alkyl Glucoside | 0.4000% |
| Polyacrylate Crosspolymer-6 | 0.2850% |
| Caprylic/Capric Triglyceride | 0.1929% |
| Caprylhydroxamic Acid | 0.1500% |
| Butyrospermum Parkii (Shea) | 0.1286% |
| Butter | |
| Cetearyl Alcohol | 0.1286% |
| Palmitic Acid | 0.1286% |
| Sodium Phytate | 0.116075% |
| Pentylene Glycol | 0.11215% |
| Allantoin | 0.1000% |
| Hedychium Coronarium Root | 0.0600% |
| Extract | |
| Acacia Senegal Gum | 0.0550% |
| Xanthan Gum | 0.0450% |
| Citric Acid | 0.0370064% |
| Arachidyl Alcohol | 0.03215% |
| Arachidyl Glucoside | 0.03215% |
| Behenyl Alcohol | 0.03215% |
| Hydrogenated Olive Oil | 0.03215% |
| Hydroxyethyl Acrylate/Sodium | 0.03215% |
| Acryloyldimethyl Taurate | |
| Copolymer | |
| Phoenix Dacgylifera (Date) Seed | 0.0200% |
| Sodium Acetylated Hyaluronate | 0.0170% |
| Cetearyl Glucoside | 0.016075% |
| Olea Europaea (Olive) Fruit Oil | 0.016075% |
| Phenethyl Alcohol | 0.016075% |
| Sodium Hyaluronate | 0.0150% |
| Ascorbic Acid | 0.0130% |
| Aloe Barbadensis Leaf Juice | 0.0100% |
| t-Butyl Alcohol* | 0.0075% |
| Sodium Hyaluronate Crosspolymer | 0.0070% |
| Glucose | 0.0056075% |
| Olea Europaea (Olive) Oil | 0.003215% |
| Unsaponifiables | |
| Sorbitan Isostearate | 0.003215% |
| Hydrolyzed Sodium Hyaluronate | 0.0030% |
| Myristic Acid | 0.0016075% |
| Stearic Acid | 0.0016075% |
| Ethylhexylglycerin | 0.0015% |
| Lauric Acid | 0.0003215% |
| Dextran | 0.0001% |
| Tripeptide-1 | 0.0001% |
| TOTAL | 100.0000% |

### Example 2: Exemplary Composition Comprising Naringenin

Table 13 provides another exemplary formulation of naringenin:

TABLE 13
Balm comprising naringenin:

| Ingredient | Concentration w/w % |
|---|---|
| Squalane | 19.5500% |
| Cetearyl Alcohol | 16.0000% |
| Caprylic/Capric Triglyceride | 12.8000% |
| C13-15 Alkane | 10.0000% |
| Simmondsia Chinensis (Jojoba) | 10.0000% |
| Seed Oil | |
| Helianthus Annuus (Sunflower) | 8.0000% |
| Seed Wax | |
| Stearyl Behenate | 6.0000% |
| C10-18-Triglycerides | 5.0000% |
| Dimethyl Isosorbide | 5.0000% |
| Polyhydroxystearic Acid | 4.0000% |
| Silica | 2.0000% |
| Glyceryl Caprylate | 1.0000% |
| Naringenin | 0.4500% |
| Ceramide NP | 0.1000% |
| Limnanthes Alba (Meadowfoam) | 0.0670% |
| Seed Oil | |
| Glyceryl Behenate | 0.0190% |
| Sodium Hyaluronate | 0.0140% |
| TOTAL | 100.0000% |

### Example 3: Exemplary Composition Comprising Naringenin

Table 14 provides another exemplary formulation of naringenin:

TABLE 14
Cream Gel comprising naringenin:

| Ingredient | Concentration w/w % |
|---|---|
| Water | 72.0442% |
| Ethoxydiglycol | 1.44675% |
| Naringenin | 0.4501% |
| Caprylic/Capric Triglyceride | 6.9554% |
| Butyrospermum Parkii (Shea) Butter | 0.1286% |
| Cetearyl Alcohol | 0.1286% |
| Palmitic Acid | 0.2286% |
| Pentylene Glycol | 0.32715% |
| Propanediol | 0.03215% |
| Hydroxyethyl Acrylate/Sodium | 0.03215% |
| Acryloyldimethyl Taurate Copolymer | |
| Hydrogenated Olive Oil | 0.03215% |
| Behenyl Alcohol | 0.03215% |
| Arachidyl Alcohol | 0.03215% |
| Arachidyl Glucoside | 0.03215% |
| Olea Europaea (Olive) Fruit Oil | 0.016075% |
| Sodium Phytate | 0.016075% |
| Cetearyl Glucoside | 0.016075% |
| Phenethyl Alcohol | 0.016075% |
| Sorbitan Isostearate | 0.003215% |
| Olea Europaea (Olive) Oil | 0.003215% |
| Unsaponifiables | |
| Stearic Acid | 0.0391075% |
| Glucose | 0.0016075% |
| Myristic Acid | 0.0016075% |
| Lauric Acid | 0.0003215% |
| Citric Acid | 0.1000064% |
| Linoleic Acid | 0.7250% |
| Lecithin | 0.6500% |
| Phytosteryl Canola Glycerides | 0.2000% |
| Oleic Acid | 0.1000% |
| Tocopherol | 0.0376% |
| Triolein | 0.0375% |
| Phospholipids | 0.3900% |
| Sphingolipids | 0.0100% |
| Sodium Citrate | 0.0050% |
| p-Anisic Acid | 0.0035% |
| Phytosterols | 0.3000% |
| C13-15 Alkane | 3.5000% |
| Butylene Glycol | 3.3000% |
| Glycerin | 2.7940% |
| Glyceryl Stearate SE | 1.7500% |
| Helianthus Annuus (Sunflower) Seed Oil | 1.5000% |
| Alteromonas Ferment Extract | 0.0100% |
| Sodium Acetylated Hyaluronate | 0.0085% |
| Sodium Hyaluronate | 0.0075% |
| Sodium Hyaluronate Crosspolymer | 0.0035% |
| Hydrolyzed Sodium Hyaluronate | 0.0015% |
| Ethylhexylglycerin | 0.00075% |
| Sodium Acrylates Copolymer | 0.7500% |
| Centella Asiatica Extract | 0.2000% |
| Cetyl Alcohol | 0.5000% |
| Hydroxyacetophenone | 0.5000% |
| 1,2-Hexanediol | 0.2500% |
| Caprylyl Glycol | 0.2500% |
| Sodium Hydroxide | 0.1000% |
| Total | 100.0000% |

### Example 4: ABTS Antioxidant Assay

The 2,2′-Azinobis [3-ethylbenzothiazoline-6-sulfonic acid]-diammonium salt (ABTS) antioxidant assay is designed to evaluate the antioxidant activity of compositions comprising naringenin in an aqueous medium. ABTS+ solution is generated as a free radical and used to measure relative ability of a compound to scavenge free radicals. Reduction of the absorbance at 734 nm was standardized to solvent control as an indication of ABTS+ scavenging.

Table 15, provided below, demonstrates the results from these experiments.

TABLE 15
ABTS (aqueous antioxidant assay) radical inhibition

| Naringenin concentration (% w/w) | % Radical Inhibition |
|---|---|
| 0.018 | 65.39 |
| 0.009 | 55.45 |
| 0.0045 | 47.50 |

As evident from the results provided in Table 15 above, compositions comprising naringenin demonstrate antioxidant activity at concentrations at low as 0.001% w/w in aqueous systems. The calculated IC₅₀ value is: 0.0058% w/w.

### Example 5: DPPH Antioxidant Activity Assay

The 2,2-diphenyl-1-picrylhydrazyl (DPPH) antioxidant assay is designed to evaluate the antioxidant activity of compositions comprising naringenin in an organic medium.

2,2-diphenyl-1-picrylhydrazyl (DPPH) is a stable free radical used to evaluate free radical scavenging capacity of a material. In the presence of an antioxidant DPPH is reduced, resulting in the formation of a colorless solution. Reduction of absorbance at 517 nm is standardized to solvent control as an indication of DPPH scavenging.

Table 16, provided below, provides the antioxidant activity data.

TABLE 16
DPPH Radical Inhibition

| Naringenin concentration (% w/w) | % Radical Inhibition |
|---|---|
| 2.00 | 46.82 |
| 1.00 | 28.44 |
| 0.50 | 18.82 |

As evident from the results provided in Table 16 above, compositions comprising naringenin demonstrate antioxidant activity at concentrations at low as 0.2% w/w in organic solvent systems.

### Example 6: Elastase Enzyme Inhibition

The assay was conducted to evaluate the elastase enzyme inhibition activity of compositions comprising naringenin.

Neutrophil infiltration and neutrophil elastase are both increased in the skin in response to UV stress. Neutrophil elastase has been shown to activate MMP-1 and MMP-2 collagenase in response to low dose UV exposure, harming the extracellular matrix and contributing to photoaging and wrinkle formation. The activity of elastase can be further upregulated in the presence of reactive oxygen species. Therefore, inhibition of this enzyme, particularly in combination with antioxidant activity, is effective in delaying wrinkle development and aiding in maintaining skin integrity. This assay is measures release of 4-nitroaniline from an Me-Suc-Ala-Ala-Pro-Val-pNA by human neutrophil elastase and can be quantified at 405 nm by comparing to solvent control.

Table 17 provides the elastase inhibition activity of compositions comprising naringenin:

TABLE 17
Elastase enzyme inhibition

| Naringenin concentration (% w/w) | % Enzyme Inhibition |
|---|---|
| 0.50 | 84.03 |
| 0.10 | 15.51 |
| 0.02 | 1.05 |

As evident from the data provide in Table 17, compositions comprising naringenin inhibits elastase enzyme activity as low at 0.02% w/w. The calculated IC₅₀ value is: 0.159% w/w.

### Example 7: Glycation Inhibition Assay

The assay was conducted to evaluate the glycation inhibition activity of compositions comprising naringenin.

Nonenzymatic glycation can occur when glucose reacts with proteins, forming a Schiff base intermediate that subsequently undergoes Amadori rearrangement to form glycosylated proteins, when they react with skin proteins to form Advanced Glycation End products (AGE). AGE formation can result in loss of skin elasticity and impaired cellular function. The glycation inhibition ability of a compound is measured by change in fluorescence m relative to solvent control.

Table 18 provides the data for glycation inhibition.

TABLE 18
Glycation Inhibition

| Naringenin concentration (% w/w) | % Enzyme Inhibition |
|---|---|
| 0.0625 | 69.05 |
| 0.031 | 60.22 |
| 0.016 | 45.49 |
| 0.008 | 27.87 |

As evident from the data provided in Table 18, compositions comprising naringenin inhibits glycation as low as 0.002% w/w. The IC₅₀ value calculated for glycation inhibition is 0.019%.

### Example 8: Pre-Clinical Assays for Measuring Anti-Inflammatory Activity

This example provides data for anti-inflammatory properties by demonstrating the reduction in inflammatory markers IL-6 and TNFα to non-stressed levels in stressed keratinocytes. Normal Human Epidermal Keratinocytes (NHEK) were stressed with a cocktail of lipopolysaccharide (LPS) and Polyinosinic-polycytidylic acid sodium salt (Poly:IC), external aggressors capable of inducing an inflammatory response. Stressed cells were subsequently treated with test articles and reduction in inflammatory markers was measuring by ELISA. Tables 19 and 20, provided below, discloses data for TNFα and IL-6 concentrations, respectively, in stressed normal human epidermal keratinocytes.

TABLE 19
TNFα concentration in stimulated (stressed)
normal human epidermal keratinocytes

| Test Article | pg/ml | % Reduction vs Treated | p-value |
|---|---|---|---|
| Unstimulated Control | 39.3 | — | 0.004135 |
| Stimulated Control | 202.6 | 0 | 1 |
| 0.01% Naringenin | 104.2 | 48.6 | 0.004838 |
| 0.007% Naringenin | 70.3 | 65.3 | 0.002961 |
| 0.0045% Naringenin | 43.8 | 78.4 | 0.002047 |
| 0.0001% Naringenin | 94.3 | 53.4 | 6.99E−07 |

TABLE 20
IL-6 concentration in stimulated (stressed)
normal human epidermal keratinocytes

| Test Article | pg/ml | % Reduction vs Treated | p-value |
|---|---|---|---|
| Unstimulated Control | 38.7 | — | 0.0029 |
| Stimulated Control | 1124.6 | 0.000 | 1 |
| 0.01% Naringenin | 76.6 | 93.2 | 0.0035 |
| 0.007% Naringenin | 50.0 | 95.6 | 0.0030 |
| 0.0045% Naringenin | 42.0 | 96.3 | 0.0029 |
| 0.0001% Naringenin | 395.8 | 64.8 | 0.0001 |

As evident from the data provided in Tables 19 and 20, compositions comprising naringenin have a robust anti-inflammatory activity.

### Example 9: Sun Protection

The assay was conducted to evaluate the sun protection factor (SPF) activity of compositions comprising naringenin. The table below provides in vitro SPF value comparison between sunscreen formulations and formulations comprising naringenin.

TABLE 21
SPF

| Formula | SPF |
|---|---|
| Sunscreen | 12.11 |
| Sunscreen + 0.45% naringenin | 14.57 |

### Example 11: Clinical Trials

The example provides a placebo-controlled double-blind study with 30 participants/group. Participants use the product twice daily and skin attributes are measured at TO, 4 weeks, and 8 weeks.

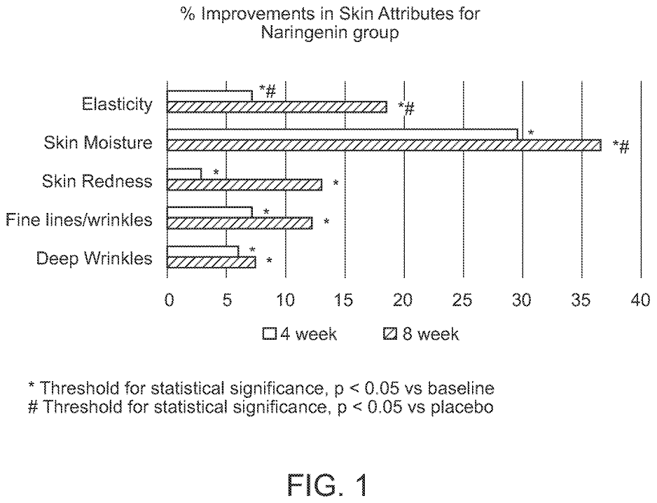

The data from the trials is provided in FIG. 1, and summarized below. The data provides:
97% of panelists showed an improvement in fine/lines wrinkles vs 52% of panelists using the placebo
97% of panelists showed an improvement in deep wrinkles vs 70% of panelists using the placebo
88% of panelists showed an improvement in skin redness vs 70% of panelists using the placebo
100% of panelists showed an improvement in skin moisturization vs 70% of panelists using the placebo
100% of panelists showed an improvement in skin elasticity vs 48% of panelists using the placebo

The results demonstrated that formula comprising naringenin was clinically proven to help moisturize and improve skin elasticity in 8 weeks, while helping to moisturize and reduce appearance of fine lines in just 4 weeks.

### Example 12: Exemplary Composition Comprising Naringenin

Table 22 provides an exemplary formulation of naringenin:

TABLE 22
Exemplary eye cream composition

| Ingredient | % w/w |
|---|---|
| Water | 72.6524% |
| Caprylic/Capric Triglyceride | 5.0000% |
| Glycerin | 3.9908% |
| Silica | 2.5600% |
| Dimethyl Isosorbide | 2.5500% |
| Squalane | 2.0000% |
| Diheptyl Succinate | 1.6200% |
| Capryloyl Glycerin/Sebacic Acid | 1.3800% |
| Copolymer | |
| Arachidyl Alcohol | 1.0800% |
| Microcrystalline Cellulose | 1.0800% |
| Hydroxyethyl Acrylate/Sodium | 0.8800% |
| Acryloyldimethyl Taurate Copolymer | |
| Behenyl Alcohol | 0.5600% |
| Hydroxyacetophenone | 0.5000% |
| Cetearyl Olivate | 0.5000% |
| Sortiban Olivate | 0.5000% |
| Hydrogenated Olive Olil | 0.4900% |
| Naringenin | 0.4500% |
| Arachidyl Glucoside | 0.3600% |
| Lauroyl Lysine | 0.3600% |
| Pentylene Glycol | 0.2950% |
| 1,2-Hexanediol | 0.2500% |
| Caprylyl Glycol | 0.2500% |
| Olea Europaea (Olive) Fruit Oil | 0.1400% |
| Disodium Acetyl Glucosamine | 0.0998% |
| Phosphate | |
| Sodium Glucuronate | 0.0998% |
| Phospholipids | 0.0900% |
| Olea Europaea (Olive) Oil | 0.0700% |
| Unsaponifiables | |
| Polysorbate 60 | 0.0600% |
| Sorbitan Isostearate | 0.0600% |
| Magnesium Sulfate | 0.0198% |
| Sodium Benzoate | 0.0110% |
| Sphingolipids | 0.0100% |
| Sodium Acetylated Hyaluronate | 0.0085% |
| Sodium Hyaluronate | 0.0075% |
| Sodium Citrate | 0.0050% |
| p-Anisic Acid | 0.0035% |
| Sodium Hyaluronate Crosspolymer | 0.0035% |
| Hydrolyzed Sodium Hyaluronate | 0.0015% |
| Acetyl Hexapeptide-8 | 0.0011% |
| Ethylhexlglycerin | 0.0008% |
| Tocopherol | 0.0001% |
| Sum Total | 100.0000% |

### Example 13: Exemplary Sunscreen Formulation

Table 23 provides an exemplary formulation of naringenin:

TABLE 23
Exemplary sunscreen formulation

| Ingredients | % w/w |
|---|---|
| Water | 42.9700% |
| Zinc Oxide | 20.0200% |
| Caprylic/Capric Triglyceride | 4.2900% |
| Dicaprylyl Carbonate | 4.0000% |
| C13-15 Alkane | 4.0000% |
| Dimethyl Isosorbide | 2.5500% |
| Propanediol | 3.7200% |
| Cetearyl Alcohol | 1.9350% |
| Neopentyl Glycol Dipheptanoate | 1.2600% |
| Polyhydroxystearic Acid | 1.0730% |
| Polyglyceryl-3 Polyricinoleate | 1.0730% |
| Isotearic Acid | 1.0730% |
| Lecithin | 1.0730% |
| Octyldodecanol | 1.0600% |
| Coco-Glucoside | 1.0500% |
| Trehalose | 1.0000% |
| Potassium Cetyl Phosphate | 1.0000% |
| Simmondsia Chinensis (jojoba) Seed Oil | 0.9998% |
| Citric Acid | 0.7770% |
| Isododecane | 0.7400% |
| Cellulose | 0.6700% |
| Caprylyl Glyceryl Ether | 0.5000% |
| Bis-Diglyceryl Polyacyladipate-2 | 0.5000% |
| Octyldodecanol | 0.5000% |
| Naringenin | 0.4500% |
| Cellulose Gum | 0.3300% |
| Disteardimonium Hectorite | 0.3000% |
| Iron Oxide (CI77492) | 0.2400% |
| Acacia Senegal Gum | 0.1650% |
| Caprylhydroxamic Acid | 0.1500% |
| Propylene Carbonate | 0.1500% |
| Xanthan Gum | 0.1350% |
| Glycerin | 0.125% |
| Sodium Phytate | 0.1000% |
| Aloe Barbadensis Leaf Juice Powder | 0.0100% |
| Glucose | 0.0060% |
| Iron Oxide (CI77491) | 0.0050% |
| Dunaliella Saline Extract | 0.0002% |
| Total | 100.0000% |

### Example 14: Exemplary Cream Formulation

Table 24 provides an exemplary formulation of naringenin:

TABLE 24
Exemplary Cream Formulations

| Ingredient | % w/w |
|---|---|
| Water | 72.0100% |
| Glycerin | 3.1250% |
| Diheptyl Succinate | 3.9200% |
| Caprylic/Capric Triglyceride | 3.0400% |
| Dimethyl Isosorbide | 2.5500% |
| Squalane | 2.0000% |
| C15-19 Alkane | 2.0000% |
| Arachidyl Alcohol | 1.0800% |
| Pseudozyma Epicola/Sunflower Seed Oil | 1.0000% |
| Ferment Extract Filtrate | |
| Propanediol | 1.2250% |
| Hydroxyethyl Acrylte/Sodium | 0.8800% |
| Acryloyldimethyl Taurate Copolymer | |
| Xylitylglucoside | 0.6000% |
| Behenyl Alcohol | 0.5600% |
| Saccharide Isomerate | 0.5100% |
| Caprylyl Glyceryl Ether | 0.5000% |
| Cetearyl Olivate | 0.5000% |
| Sortiban Olivate | 0.5000% |
| Hydrogenated Olive Oil | 0.4900% |
| Anhydroxylitol | 0.4200% |
| Naringenin | 0.4500% |
| Arachidyl Glucoside | 0.3600% |
| Phospholipids | 0.3200% |
| Phytosterols | 0.3200% |
| Linoleic Acid | 0.3200% |
| Ceramide NP | 0.2050% |
| Propylene Glycol | 0.1900% |
| Xylitol | 0.1800% |
| Caprylhydroxamic Acid | 0.1500% |
| Olea Europaea (Olive) Fruit Oil | 0.1400% |
| Capryloyl Glycerin/Sebacic Acid | 0.0800% |
| Copolymer | |
| Olea Europaea (Olive) Oil | 0.0700% |
| Unsaponifiables | |
| Glucose | 0.0600% |
| Polysorbate 60 | 0.0600% |
| Sorbitan Isostearate | 0.0600% |
| Hydrogenated Lecithin | 0.0600% |
| Glyceryl Stearate | 0.0250% |
| Citric Acid | 0.0100% |
| Sodium Citrate | 0.0100% |
| Ceramide NS | 0.0050% |
| Ceramide AG | 0.0050% |
| Ceramide AP | 0.0050% |
| Cholesterol | 0.0050% |
| Ceramide EOP | 0.0001% |
| Total | 100.000% |

### Example 14: Exemplary Night Mask Formulation

Table 25 provides an exemplary formulation of naringenin:

TABLE 25
Exemplary Night Mask Formulation:

| Ingredients | % w/w |
|---|---|
| Water | 58.8338% |
| Gluconolactone | 10.0000% |
| Squalane | 4.7500% |
| Glycerin | 4.5000% |
| Propanediol | 3.0000% |
| Hemisqualane | 3.0000% |
| Neopentyl Glycol Diheptanoate | 2.6000% |
| Dimethyl Isosorbide | 2.5500% |
| Sodium Polyacryloyldimethyl Taurate | 2.0000% |
| Polyglyceryl-3Methylglucose Distearate | 2.0000% |
| Isododecane | 1.4000% |
| Hydroxyethyl Acrylate/Sodium Acryloyl | 1.0000% |
| Dimethyl Taurate Copolymer | |
| Helianthus Annuus Seed Oil | 0.6930% |
| Sodium Hydroxide | 0.8750% |
| Hydroxyacetophenone | 0.5000% |
| Naringenin | 0.4500% |
| Inulin Lauryl Carbamate | 0.5000% |
| Persea Gratissima (Avocado) Fruit Extract | 0.3000% |
| Maltodextrin | 0.3000% |
| Caprylyl Glycol | 0.3000% |
| 1,2-Hexanediol | 0.2000% |
| Hydrolyzed Jojoba Esters | 0.2000% |
| Pentylene Glycol | 0.0200% |
| Sodium Acetylated Hyaluronate | 0.0085% |
| Sodium Hyaluronate | 0.0075% |
| Capsicum Annuum Fruit Extract | 0.0065% |
| Sodium Hyaluronate Crosspolymer | 0.0035% |
| Hydrolyzed Sodium Hyaluronate | 0.0015% |
| Ethylhexlglycerin | 0.0008% |
| Total | 100% |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A composition for topical administration comprising an effective amount of naringenin.

2. The composition of claim 1, wherein the composition comprises an emulsion.

3. The composition of claim 2, wherein the emulsion is an oil/water emulsion.

4. The composition of claim 1, wherein the composition further comprises a solvent.

5. The composition of claim 4, wherein the solvent is a cosmetic solvent.

6. The composition of claim 4, wherein the solvent is selected from a group consisting of: dimethyl isosorbide, ethoxydiglycol, isopropyl lauryl sarcosinate, glycols, and any combination thereof.

7. The composition of claim 6, wherein the solvent is dimethyl isosorbide.

8. The composition of claim 6, wherein the solvent is ethoxydiglycol.

9. The composition of claim 6, wherein the solvent is isopropyl lauryl sarcosinate.

10. The composition of claim 6, where in the solvent is a glycol.

11. The composition of claim 10, wherein the glycol is selected from a group consisting of pentylene glycol, butylene glycol, and any combination thereof.

12. The composition of claim 4, wherein the solvent is selected from a group consisting of: phenylpropanol, 1,2-hexanediol, propanediol, isopentyldiol, 1,2 heptanediol, and any combination thereof.

13. The composition of claim 1, which is stable under storage for at least two (2) years under ambient conditions.

14. The composition of claim 1, which is stable under storage for at least one (1) year under ambient conditions.

15. The composition of claim 1, which is stable under storage for at least six (6) months under ambient conditions.

16. The composition of claim 1, which is stable under storage for at least three (3) months under ambient conditions

17. The composition of claim 1, wherein the composition demonstrates has less than about 10% degradation when stored at 70° C. for one (1) month.

18. The composition of claim 1, wherein naringenin is present in a concentration of about 0.0001% to about 5.0% w/w.

19. The composition of claim 1, wherein naringenin is present in a concentration of about 0.0001% to about 1.0% w/w.

20. The composition of claim 1, wherein naringenin is present in a concentration of about 0.001% to about 0.5% w/w.

21. The composition of claim 1, wherein naringenin is present in a concentration of about 0.01% to about 0.5% w/w.

22. The composition of claim 1, wherein naringenin is present in a concentration of about 0.1% to about 0.5% w/w.

23. The composition of claim 1, wherein naringenin is present in a concentration of about 0.45% w/w.

24. The composition of claim 1, wherein the composition is selected from a group consisting of: fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, hair treatment, and any combination thereof.

25. The composition of claim 1, wherein the composition further comprises one or more excipients selected from a group consisting of; solvent, emulsifier, preservative, antioxidant, emollient, thickening agent, penetration enhancer, surfactant, diluent, filler, carrier, and/or pH control agent.

26. The composition of claim 25, wherein the preservative is an antimicrobial preservative or chelating agent.

27. The composition of claim 1, wherein the composition further comprises one or more excipients selected from a group consisting of: water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and ethylhexylglycerin.

28. The composition of claim 1, wherein the composition further comprises one or more excipients selected from a group consisting of: water, glycerin, niacinamide, squalane, C₁₄₋₂₂ alcohols, oil, ethoxydiglycol, propanediol, caprylyl glyceryl ether, lauroyl lysine, panthenol, butylene glycol, C₁₂₋₂₀ alkyl glucoside, polyacrylate crosspolymer-6, caprylic/capric triglyceride, caprylhydroxamic acid, shea butter, cetearyl alcohol, palmitic acid, sodium phytate, pentylene glycol, allantoin, Hedychium coronarium root extract, Acacia senegal gum, xantham gum, citric acid, arachidyl alcohol, arachidyl glucoside, behenyl alcohol, hydrogenated olive oil, hydroxyethyl acrylate/sodium acryloyldimethyl taurate copolymer, date seed, sodium acetylated hyaluronate, cetearyl glucoside, olive oil, phenethyl alcohol, sodium hyaluronate, ascorbic acid, Aloe leaf juice, t-butyl alcohol, sodium hyaluronate crosspolymer, glucose, sorbitan isostearate, hydrolyzed sodium hyaluronate, myristic acid, stearic acid, ethylhexylglycerin, lauric acid, dextran, and tripeptide-1.

29. The composition of claim 1, wherein the composition further comprises one or more excipients selected from a group consisting of: squalane, cetearyl alcohol, caprylic/capric triglyceride, C₁₃₋₁₅ alkane, seed oil, seed wax, stearyl behenate, C₁₀₋₁₈ triglycerides, dimethyl isosorbide, polyhydroxystearic acid, silica, glyceryl caprylate, ceramide, glyceryl behenate, and sodium hyaluronate.

30. The composition of claim 1, wherein the composition further comprises one or more excipients selected from a group consisting of: water, ethoxydiglycol, caprylic/capric triglyceride, shea butter, cetearyl alcohol, palmitic acid, pentylene glycol, propanediol, hydroxyethyl acrylate/sodium acryloyldimethyl taurate copolymer, hydrogenated olive oil, behenyl alcohol, arachidyl alcohol, arachidyl glucoside, olive oil, sodium phytate, cetearyl glucoside, phenethyl alcohol, sorbitan isostearate, stearic acid, glucose, myristic acid, lauric acid, citric acid, linoleic acid, lecithin, phytosteryl canola glycerides, oleic acid, tocopherol, triolein, phospholipids, sphingolipids, sodium citrate, p-anisic acid, phytosterols, C₁₃₋₁₅ alkane, butylene glycol, glycerin, glyceryl stearate, alteromanas ferment extract, sodium acetylated hyaluronate, sodium hyaluronate, sodium hyaluronate crosspolymer, hydrolyzed sodium hyaluronate, ethylhexylglycerin, sodium acrylates copolymer, Centella asiatica extract, cetyl alcohol, hydroxyacetophenone, 1,2, hexanediol, caprylyl glycol, and sodium hydroxide.

31. The composition of claim 1, wherein the composition is:

| Ingredient | Concentration w/w % |
|---|---|
| Water | 30%-90% |
| Glycerin | 2-10% |
| Niacinamide | 0-10% |
| Squalane | 0-10% |
| C₁₄₋₂₂ Alcohols | 0-5% |
| Simmondsia Chinensis (Jojoba) | 0-5% |
| Seed Oil | |
| Ethoxydiglycol | 0-5% |
| Propanediol | 0-5% |
| Caprylyl Glyceryl Ether | 0-2% |
| Lauroyl Lysine | 0-2% |
| Panthenol | 0-2% |
| Butylene Glycol | 0-2% |
| Naringenin | 0.0001-5.00% |
| C12-20 Alkyl Glucoside | 0-2% |
| Polyacrylate Crosspolymer-6 | 0-2% |
| Caprylic/Capric Triglyceride | 0-2% |
| Caprylhydroxamic Acid | 0-2% |
| Butyrospermum Parkii (Shea) | 0-2% |
| Butter | |
| Cetearyl Alcohol | 0-2% |
| Palmitic Acid | 0-2% |
| Sodium Phytate | 0-2% |
| Pentylene Glycol | 0-2% |
| Allantoin | 0-2% |
| Hedychium Coronarium Root | 0-1% |
| Extract | |
| Acacia Senegal Gum | 0-1% |
| Xanthan Gum | 0-1% |
| Citric Acid | 0-1% |
| Arachidyl Alcohol | 0-1% |
| Arachidyl Glucoside | 0-1% |
| Behenyl Alcohol | 0-1% |
| Hydrogenated Olive Oil | 0-1% |
| Hydroxyethyl Acrylate/Sodium | 0-1% |
| Acryloyldimethyl Taurate | |
| Copolymer | |
| Phoenix Dacgylifera (Date) Seed | 0-0.5% |
| Sodium Acetylated Hyaluronate | 0-0.5% |
| Cetearyl Glucoside | 0-0.5% |
| Olea Europaea (Olive) Fruit Oil | 0-0.5% |
| Phenethyl Alcohol | 0-0.5% |
| Sodium Hyaluronate | 0-0.5% |
| Ascorbic Acid | 0-0.5% |
| Aloe Barbadensis Leaf Juice | 0-0.5% |
| t-Butyl Alcohol* | 0-0.1% |
| Sodium Hyaluronate Crosspolymer | 0-0.1% |
| Glucose | 0-0.1% |
| Olea Europaca (Olive) Oil | 0-0.1% |
| Unsaponifiables | |
| Sorbitan Isostearate | 0-0.1% |
| Hydrolyzed Sodium Hyaluronate | 0-0.1% |
| Myristic Acid | 0-0.1% |
| Stearic Acid | 0-0.1% |
| Ethylhexylglycerin | 0-0.1% |
| Lauric Acid | 0-0.1% |
| Dextran | 0-0.1% |
| Tripeptide-1 | 0-0.1% |
| Total | 100% |

32. The composition of claim 1, wherein the composition is:

| Ingredient | Concentration w/w % |
|---|---|
| Squalane | 5-40% |
| Cetearyl Alcohol | 5-40% |
| Caprylic/Capric Triglyceride | 2-25% |
| C13-15 Alkane | 2-25% |
| Simmondsia Chinensis (Jojoba) | 2-30% |
| Seed Oil | |
| Helianthus Annuus (Sunflower) | 2-20% |
| Seed Wax | |
| Stearyl Behenate | 2-15% |
| C10-18-Triglycerides | 1-15% |
| Dimethyl Isosorbide | 1-15% |
| Polyhydroxystearic Acid | 0-10% |
| Silica | 0-10% |
| Glyceryl Caprylate | 0-5% |
| Naringenin | 0.0001-5.00% |
| Ceramide NP | 0-2% |
| Limnanthes Alba (Meadowfoam) | 0-2% |
| Seed Oil | |
| Glyceryl Behenate | 0-2% |
| Sodium Hyaluronate | 0-20% |
| TOTAL | 100.0000% |

33. The composition of claim 1, wherein the composition is:

| Ingredient | Concentration w/w % |
|---|---|
| Water | 40-90% |
| Ethoxydiglycol | 0-10% |
| Naringenin | 0.0001-5.00% |
| Caprylic/Capric Triglyceride | 2-20% |
| Butyrospermum Parkii (Shea) Butter | 0-5% |
| Cetcaryl Alcohol | 0-5% |
| Palmitic Acid | 0-5% |
| Pentylene Glycol | 0-5% |
| Propanediol | 0-2% |
| Hydroxyethyl Acrylate/Sodium | 0-2% |
| Acryloyldimethyl Taurate | |
| Copolymer | |
| Hydrogenated Olive Oil | 0-2% |
| Behenyl Alcohol | 0-2% |
| Arachidyl Alcohol | 0-2% |
| Arachidyl Glucoside | 0-2% |
| Olea Europaea (Olive) Fruit Oil | 0-2% |
| Sodium Phytate | 0-2% |
| Cetearyl Glucoside | 0-2% |
| Phenethyl Alcohol | 0-2% |
| Sorbitan Isostearate | 0-2% |
| Olea Europaea (Olive) Oil | 0-2% |
| Unsaponifiables | |
| Stearic Acid | 0-2% |
| Glucose | 0-1% |
| Myristic Acid | 0-0.1% |
| Lauric Acid | 0-0.1% |
| Citric Acid | 0-2% |
| Linoleic Acid | 0-3% |
| Lecithin | 0-3% |
| Phytosteryl Canola Glycerides | 0-2% |
| Oleic Acid | 0-2% |
| Tocopherol | 0-1% |
| Triolein | 0-1% |
| Phospholipids | 0-5% |
| Sphingolipids | 0-1% |
| Sodium Citrate | 0-1% |
| p-Anisic Acid | 0-0.1% |
| Phytosterols | 0-5% |
| C13-15 Alkane | 0-10% |
| Butylene Glycol | 0-10% |
| Glycerin | 0-10% |
| Glyceryl Stearate SE | 0-10% |
| Helianthus Annuus (Sunflower) | 0-10% |
| Seed Oil | |
| Alteromonas Ferment Extract | 0-2% |
| Sodium Acetylated Hyaluronate | 0-0.1% |
| Sodium Hyaluronate | 0-0.1% |
| Sodium Hyaluronate Crosspolymer | 0-0.1% |
| Hydrolyzed Sodium Hyaluronate | 0-0.1% |
| Ethylhexylglycerin | 0.00075% |
| Sodium Acrylates Copolymer | 0-5% |
| Centella Asiatica Extract | 0-5% |
| Cetyl Alcohol | 0-5% |
| Hydroxyacetophenone | 0-5% |
| 1,2-Hexanediol | 0-5% |
| Caprylyl Glycol | 0-5% |
| Sodium Hydroxide | 0-2% |
| Total | 100.0000% |

34. The composition of claim 1, wherein the composition is:

| Ingredient | % w/w |
|---|---|
| Water | 40-90% |
| Caprylic/Capric Triglyceride | 2-20% |
| Glycerin | 1-15% |
| Silica | 1-10% |
| Dimethyl Isosorbide | 1-10% |
| Squalane | 0.5-5% |
| Diheptyl Succinate | 0.4-4% |
| Capryloyl Glycerin/Sebacic Acid | 0.5-5% |
| Copolymer | |
| Arachidyl Alcohol | 0.5-5% |
| Microcrystalline Cellulose | 0.2-5% |
| Hydroxyethyl Acrylate/Sodium | 0.2-5% |
| Acryloyldimethyl Taurate Copolymer | |
| Behenyl Alcohol | 0.2-5% |
| Hydroxyacetophenone | 0.2-5% |
| Cetearyl Olivate | 0.2-5% |
| Sortiban Olivate | 0.2-5% |
| Hydrogenated Olive Olil | 0.2-5% |
| Naringenin | 0.2-5% |
| Arachidyl Glucoside | 0.2-2% |
| Lauroyl Lysine | 0.1-2% |
| Pentylene Glycol | 0.1-2% |
| 1,2-Hexanediol | 0.1-2% |
| Caprylyl Glycol | 0.1-2% |
| Olea Europaea (Olive) Fruit Oil | 0.05-1% |
| Disodium Acetyl Glucosamine | 0.02-0.5% |
| Phosphate | |
| Sodium Glucuronate | 0.02-0.5% |
| Phospholipids | 0.02-0,5% |
| Olea Europaea (Olive) Oil | 0.01-0.2% |
| Unsaponifiables | |
| Polysorbate 60 | 0.01-0.2% |
| Sorbitan Isostearate | 0.01-0.2% |
| Magnesium Sulfate | 0.005-0.1% |
| Sodium Benzoate | 0.005-0.1% |
| Sphingolipids | 0.005-0.1% |
| Sodium Acetylated Hyaluronate | 0.005-0.1% |
| Sodium Hyaluronate | 0.005-0.1% |
| Sodium Citrate | 0.0025-0.05% |
| p-Anisic Acid | 0.0015-0.05% |
| Sodium Hyaluronate Crosspolymer | 0.0015-0.05% |
| Hydrolyzed Sodium Hyaluronate | 0.00015-0.005% |
| Acetyl Hexapeptide-8 | 0.00015-0.005% |
| Ethylhexlglycerin | 0.00015-0.005% |
| Tocopherol | 0.000015-0.0005% |
| Sum Total | 100.0000% |

35. The composition of claim 1, wherein the composition is:

| Ingredients | % w/w |
|---|---|
| Water | 25-80% |
| Zinc Oxide | 5-40% |
| Caprylic/Capric Triglyceride | 1-20% |
| Dicaprylyl Carbonate | 1-20% |
| C13-15 Alkane | 1-20% |
| Dimethyl Isosorbide | 0.5-10% |
| Propanediol | 0.5-10% |
| Cetearyl Alcohol | 0.25-5% |
| Neopentyl Glycol Dipheptanoate | 0.25-5% |
| Polyhydroxystearic Acid | 0.25-5% |
| Polyglyceryl-3 Polyricinoleate | 0.25-5% |
| Isotearic Acid | 0.25-5% |
| Lecithin | 0.25-5% |
| Octyldodecanol | 0.25-5% |
| Coco-Glucoside | 0.25-5% |
| Trehalose | 0.25-5% |
| Potassium Cetyl Phosphate | 0.25-5% |
| Simmondsia Chinensis (jojoba) Seed Oil | 0.25-5% |
| Citric Acid | 0.1-5% |
| Isododecane | 0.1-5% |
| Cellulose | 0.1-5% |
| Caprylyl Glyceryl Ether | 0.1-5% |
| Bis-Diglyceryl Polyacyladipate-2 | 0.1-5% |
| Octyldodecanol | 0.1-5% |
| Naringenin | 0.1-5% |
| Cellulose Gum | 0.1-5% |
| Disteardimonium Hectorite | 0.1-5% |
| Iron Oxide (CI77492) | 0.2400% |
| Acacia Senegal Gum | 0.05-1% |
| Caprylhydroxamic Acid | 0.05-1% |
| Propylene Carbonate | 0.05-1% |
| Xanthan Gum | 0.05-1% |
| Glycerin | 0.05-1% |
| Sodium Phytate | 0.05-1% |
| Aloe Barbadensis Leaf Juice Powder | 0.005-0.2% |
| Glucose | 0.001-0.05% |
| Iron Oxide (CI77491) | 0.001-0.05% |
| Dunaliella Saline Extract | 0.0005-0.005% |
| Total | 100.0000% |

36. The composition of claim 1, wherein the composition is:

| Ingredient | % w/w |
|---|---|
| Water | 40-90% |
| Glycerin | 1-10% |
| Diheptyl Succinate | 1-10% |
| Caprylic/Capric Triglyceride | 1-10% |
| Dimethyl Isosorbide | 0.5-5% |
| Squalane | 0.5-5% |
| C1-19 Alkane | 0.5-5% |
| Arachidyl Alcohol | 0.1-5% |
| Pseudozyma Epicola/Sunflower Seed Oil | 0.1-5% |
| Ferment Extract Filtrate | |
| Propanediol | 0.1-5% |
| Hydroxyethyl Acrylte/Sodium | 0.1-5% |
| Acryloyldimethyl Taurate Copolymer | |
| Xylitylglucoside | 0.1-5% |
| Behenyl Alcohol | 0.1-2.5% |
| Saccharide Isomerate | 0.1-2.5% |
| Caprylyl Glyceryl Ether | 0.1-2.5% |
| Cetearyl Olivate | 0.1-2.5% |
| Sortiban Olivate | 0.1-2.5% |
| Hydrogenated Olive Oil | 0.1-2.5% |
| Anhydroxylitol | 0.1-2.5% |
| Naringenin | 0.1-5% |
| Arachidyl Glucoside | 0.1-2.5% |
| Phospholipids | 0.1-2.5% |
| Phytosterols | 0.1-2.5% |
| Linoleic Acid | 0.1-2.5% |
| Ceramide NP | 0.1-2.5% |
| Propylene Glycol | 0.05-2% |
| Xylitol | 0.05-2% |
| Caprylhydroxamic Acid | 0.05-2% |
| Olea Europaea (Olive) Fruit Oil | 0.05-2% |
| Capryloyl Glycerin/Sebacic Acid | 0.01-2% |
| Copolymer | |
| Olea Europaea (Olive) Oil | 0.01-1% |
| Unsaponifiables | |
| Glucose | 0.01-1% |
| Polysorbate 60 | 0.01-1% |
| Sorbitan Isostearate | 0.01-1% |
| Hydrogenated Lecithin | 0.01-1% |
| Glyceryl Stearate | 0.005-0.5% |
| Citric Acid | 0.005-0.5% |
| Sodium Citrate | 0.001-0.5% |
| Ceramide NS | 0.001-0.5% |
| Ceramide AG | 0.001-0.5% |
| Ceramide AP | 0.001-0.5% |
| Cholesterol | 0.001-0.5% |
| Ceramide EOP | 0.00001-0.005% |
| Total | 100.000% |

37. The composition of claim 1, wherein the composition is:

| Ingredients | % w/w |
|---|---|
| Water | 40-80% |
| Gluconolactone | 2.5-25% |
| Squalane | 1-10% |
| Glycerin | 1-10% |
| Propanediol | 1-10% |
| Hemisqualane | 1-10% |
| Neopentyl Glycol Diheptanoate | 0.5-5% |
| Dimethyl Isosorbide | 0.5-5% |
| Sodium Polyacryloyldimethyl Taurate | 0.5-5% |
| Polyglyceryl-3Methylglucose Distearate | 0.5-5% |
| Isododecane | 0.5-5% |
| Hydroxyethyl Acrylate/Sodium Acryloyl | 0.1-2.5% |
| Dimethyl Taurate Copolymer | |
| Helianthus Annuus Seed Oil | 0.1-2.5% |
| Sodium Hydroxide | 0.1-2.5% |
| Hydroxyacetophenone | 0.1-2.5% |
| Naringenin | 0.1-5% |
| Inulin Lauryl Carbamate | 0.1-5% |
| Persea Gratissima (Avocado) Fruit Extract | 0.05-2.5% |
| Maltodextrin | 0.05-2.5% |
| Caprylyl Glycol | 0.05-2.5% |
| 1,2-Hexanediol | 0.05-2.5% |
| Hydrolyzed Jojoba Esters | 0.05-2.5% |
| Pentylene Glycol | 0.05-2.5% |
| Sodium Acetylated Hyaluronate | 0.005-0.25% |
| Sodium Hyaluronate | 0.0025-0.25% |
| Capsicum Annuum Fruit Extract | 0.0015-0.05% |
| Sodium Hyaluronate Crosspolymer | 0.0015-0.05% |
| Hydrolyzed Sodium Hyaluronate | 0.00015-0.005% |
| Ethylhexlglycerin | 0.0001-0.01% |
| Total | 100% |

38. The composition of claim 1, wherein naringenin is encapsulated.

39. A method of treating or preventing a topical condition in a subject by administration of a composition comprising an effective amount of naringenin.

40. The method of claim 39, wherein the administration of the composition prevents fine lines or wrinkles on skin of the subject.

41. The method of claim 39, wherein the administration of the composition leads to supporting firmness and elasticity of skin of the subject.

42. The method of claim 39, wherein the administration of the composition leads to the prevention of skin and hair damaged by reactive oxygen species.

43. The method of claim 39, wherein the administration of the composition leads to protection from environment aggressors on skin.

44. The method of claim 39, wherein the administration of the composition leads to supporting skin elasticity.

45. The method of claim 39, wherein the topical condition is glycation.

46. The method of claim 39, wherein the administration of the composition leads increase in skin and/or hair resiliency.

47. The method of claim 39, wherein the administration of the composition leads to increase in elasticity of skin.

48. The method of claim 39, wherein the administration of the composition leads to prevention of skin damage.

49. The method of claim 39, wherein the composition is administered topically.

50. The method of claim 39, wherein the composition comprises an emulsion.

51. The method of claim 39, wherein the emulsion is an oil/water emulsion.

52. The method of claim 39, wherein the composition further comprises a solvent.

53. The method of claim 39, wherein the solvent is a cosmetic solvent.

54. The method of claim 39, wherein the solvent is selected from a group consisting of: dimethyl isosorbide, ethoxydiglycol, isopropyl lauryl sarcosinate, glycols, and any combination thereof.

55. The method of claim 52, wherein the solvent is dimethyl isosorbide.

56. The method of claim 52, wherein the solvent is ethoxydiglycol.

57. The method of claim 52, wherein the solvent is isopropyl lauryl sarcosinate.

58. The method of claim 52, where in the solvent is a glycol.

59. The method of claim 58, wherein the glycol is selected from a group consisting of pentylene glycol, butylene glycol, and any combination thereof.

60. The method of claim 52, wherein the solvent is selected from a group consisting of: phenylpropanol, 1,2-hexanediol, propanediol, isopentyldiol, 1,2 heptanediol, and any combination thereof.

61. The method of claim 39, which is stable under storage for at least two (2) years under ambient conditions.

62. The method of claim 39, which is stable under storage for at least one (1) year under ambient conditions.

63. The method of claim 39, which is stable under storage for at least six (6) months under ambient conditions.

64. The method of claim 39, which is stable under storage for at least three (3) months under ambient conditions

65. The method of claim 39, wherein the composition demonstrates has less than about 10% degradation when stored at 70° C. for one (1) month.

66. The method of claim 39, wherein naringenin is present in a concentration of about 0.0001% to about 5.0% w/w.

67. The method of claim 39, wherein naringenin is present in a concentration of about 0.0001% to about 1.0% w/w.

68. The method of claim 39, wherein naringenin is present in a concentration of about 0.001% to about 0.5% w/w.

69. The method of claim 39, wherein naringenin is present in a concentration of about 0.01% to about 0.5% w/w.

70. The method of claim 39, wherein naringenin is present in a concentration of about 0.1% to about 0.5% w/w.

71. The method of claim 39, wherein naringenin is present in a concentration of about 0.45% w/w.

72. The method of claim 39, wherein the composition is selected from a group consisting of: fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, hair treatment, and any combination thereof.

73. The method of claim 39, wherein the composition further comprises one or more excipients selected from a group consisting of: solvent, emulsifier, preservative, antioxidant, emollient, thickening agent, penetration enhancer, surfactant, diluent, filler, carrier, and/or pH control agent.

74. The method of claim 73, wherein the preservative is an antimicrobial preservative or chelating agent.

75. The method of claim 39, wherein the composition further comprises one or more excipients selected from a group consisting of: water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and ethylhexylglycerin.

76. The method of claim 39, wherein the composition further comprises one or more excipients selected from a group consisting of: water, glycerin, niacinamide, squalane, C₁₄₋₂₂ alcohols, oil, ethoxydiglycol, propanediol, caprylyl glyceryl ether, lauroyl lysine, panthenol, butylene glycol, C₁₂₋₂₀ alkyl glucoside, polyacrylate crosspolymer-6, caprylic/capric triglyceride, caprylhydroxamic acid, shea butter, cetearyl alcohol, palmitic acid, sodium phytate, pentylene glycol, allantoin, Hedychium coronarium root extract, Acacia senegal gum, xantham gum, citric acid, arachidyl alcohol, arachidyl glucoside, behenyl alcohol, hydrogenated olive oil, hydroxyethyl acrylate/sodium acryloyldimethyl taurate copolymer, date seed, sodium acetylated hyaluronate, cetearyl glucoside, olive oil, phenethyl alcohol, sodium hyaluronate, ascorbic acid, Aloe leaf juice, t-butyl alcohol, sodium hyaluronate crosspolymer, glucose, sorbitan isostearate, hydrolyzed sodium hyaluronate, myristic acid, stearic acid, ethylhexylglycerin, lauric acid, dextran, and tripeptide-1.

77. The method of claim 39, wherein the composition further comprises one or more excipients selected from a group consisting of: squalane, cetearyl alcohol, caprylic/capric triglyceride, C₁₃₋₁₅ alkane, seed oil, seed wax, stearyl behenate, C₁₀₋₁₈ triglycerides, dimethyl isosorbide, polyhydroxystearic acid, silica, glyceryl caprylate, ceramide, glyceryl behenate, and sodium hyaluronate.

78. The method of claim 39, wherein the composition further comprises one or more excipients selected from a group consisting of: water, ethoxydiglycol, caprylic/capric triglyceride, shea butter, cetearyl alcohol, palmitic acid, pentylene glycol, propanediol, hydroxyethyl acrylate/sodium acryloyldimethyl taurate copolymer, hydrogenated olive oil, behenyl alcohol, arachidyl alcohol, arachidyl glucoside, olive oil, sodium phytate, cetearyl glucoside, phenethyl alcohol, sorbitan isostearate, stearic acid, glucose, myristic acid, lauric acid, citric acid, linoleic acid, lecithin, phytosteryl canola glycerides, oleic acid, tocopherol, triolein, phospholipids, sphingolipids, sodium citrate, p-anisic acid, phytosterols, C₁₃₋₁₅ alkane, butylene glycol, glycerin, glyceryl stearate, alteromanas ferment extract, sodium acetylated hyaluronate, sodium hyaluronate, sodium hyaluronate crosspolymer, hydrolyzed sodium hyaluronate, ethylhexylglycerin, sodium acrylates copolymer, Centella asiatica extract, cetyl alcohol, hydroxyacetophenone, 1,2, hexanediol, caprylyl glycol, and sodium hydroxide.

79. The method of claim 39, wherein the composition is:

| Ingredient | Concentration w/w % |
|---|---|
| Water | 30%-90% |
| Glycerin | 2-10% |
| Niacinamide | 0-10% |
| Squalane | 0-10% |
| C₁₄₋₂₂ Alcohols | 0-5% |
| Simmondsia Chinensis (Jojoba) | 0-5% |
| Seed Oil | |
| Ethoxydiglycol | 0-5% |
| Propanediol | 0-5% |
| Caprylyl Glyceryl Ether | 0-2% |
| Lauroyl Lysine | 0-2% |
| Panthenol | 0-2% |
| Butylene Glycol | 0-2% |
| Naringenin | 0.0001-5.00% |
| C12-20 Alkyl Glucoside | 0-2% |
| Polyacrylate Crosspolymer-6 | 0-2% |
| Caprylic/Capric Triglyceride | 0-2% |
| Caprylhydroxamic Acid | 0-2% |
| Butyrospermum Parkii (Shea) | 0-2% |
| Butter | |
| Cetearyl Alcohol | 0-2% |
| Palmitic Acid | 0-2% |
| Sodium Phytate | 0-2% |
| Pentylene Glycol | 0-2% |
| Allantoin | 0-2% |
| Hedychium Coronarium Root | 0-1% |
| Extract | |
| Acacia Senegal Gum | 0-1% |
| Xanthan Gum | 0-1% |
| Citric Acid | 0-1% |
| Arachidyl Alcohol | 0-1% |
| Arachidyl Glucoside | 0-1% |
| Behenyl Alcohol | 0-1% |
| Hydrogenated Olive Oil | 0-1% |
| Hydroxyethyl Acrylate/Sodium | 0-1% |
| Acryloyldimethyl Taurate | |
| Copolymer | |
| Phoenix Dacgylifera (Date) Seed | 0-0.5% |
| Sodium Acetylated Hyaluronate | 0-0.5% |
| Cetearyl Glucoside | 0-0.5% |
| Olea Europaea (Olive) Fruit Oil | 0-0.5% |
| Phenethyl Alcohol | 0-0.5% |
| Sodium Hyaluronate | 0-0.5% |
| Ascorbic Acid | 0-0.5% |
| Aloe Barbadensis Leaf Juice | 0-0.5% |
| t-Butyl Alcohol* | 0-0.1% |
| Sodium Hyaluronate Crosspolymer | 0-0.1% |
| Glucose | 0-0.1% |
| Olea Europaea (Olive) Oil | 0-0.1% |
| Unsaponifiables | |
| Sorbitan Isostearate | 0-0.1% |
| Hydrolyzed Sodium Hyaluronate | 0-0.1% |
| Myristic Acid | 0-0.1% |
| Stearic Acid | 0-0.1% |
| Ethylhexylglycerin | 0-0.1% |
| Lauric Acid | 0-0.1% |
| Dextran | 0-0.1% |
| Tripeptide-1 | 0-0.1% |
| Total | 100% |

80. The method of claim 39, wherein the composition is:

| Ingredient | Concentration w/w % |
|---|---|
| Squalane | 5-40% |
| Cetearyl Alcohol | 5-40% |
| Caprylic/Capric Triglyceride | 2-25% |
| C13-15 Alkane | 2-25% |
| Simmondsia Chinensis (Jojoba) Seed Oil | 2-30% |
| Helianthus Annuus (Sunflower) Seed | 2-20% |
| Wax | |
| Stearyl Behenate | 2-15% |
| C10-18-Triglycerides | 1-15% |
| Dimethyl Isosorbide | 1-15% |
| Polyhydroxystearic Acid | 0-10% |
| Silica | 0-10% |
| Glyceryl Caprylate | 0-5% |
| Naringenin | 0.0001-5.00% |
| Ceramide NP | 0-2% |
| Limnanthes Alba (Meadowfoam) Seed | 0-2% |
| Oil | |
| Glyceryl Behenate | 0-2% |
| Sodium Hyaluronate | 0-20% |
| TOTAL | 100.0000% |

81. The method of claim 39, wherein the composition is:

| Ingredient | Concentration w/w % |
|---|---|
| Water | 40-90% |
| Ethoxydiglycol | 0-10% |
| Naringenin | 0.0001-5.00% |
| Caprylic/Capric Triglyceride | 2-20% |
| Butyrospermum Parkii (Shea) Butter | 0-5% |
| Cetearyl Alcohol | 0-5% |
| Palmitic Acid | 0-5% |
| Pentylene Glycol | 0-5% |
| Propanediol | 0-2% |
| Hydroxyethyl Acrylate/Sodium | 0-2% |
| Acryloyldimethyl Taurate | |
| Copolymer | |
| Hydrogenated Olive Oil | 0-2% |
| Behenyl Alcohol | 0-2% |
| Arachidyl Alcohol | 0-2% |
| Arachidyl Glucoside | 0-2% |
| Olea Europaea (Olive) Fruit Oil | 0-2% |
| Sodium Phytate | 0-2% |
| Cetearyl Glucoside | 0-2% |
| Phenethyl Alcohol | 0-2% |
| Sorbitan Isostearate | 0-2% |
| Olea Europaea (Olive) Oil | 0-2% |
| Unsaponifiables | |
| Stearic Acid | 0-2% |
| Glucose | 0-1% |
| Myristic Acid | 0-0.1% |
| Lauric Acid | 0-0.1% |
| Citric Acid | 0-2% |
| Linoleic Acid | 0-3% |
| Lecithin | 0-3% |
| Phytosteryl Canola Glycerides | 0-2% |
| Oleic Acid | 0-2% |
| Tocopherol | 0-1% |
| Triolein | 0-1% |
| Phospholipids | 0-5% |
| Sphingolipids | 0-1% |
| Sodium Citrate | 0-1% |
| p-Anisic Acid | 0-0.1% |
| Phytosterols | 0-5% |
| C13-15 Alkane | 0-10% |
| Butylene Glycol | 0-10% |
| Glycerin | 0-10% |
| Glyceryl Stearate SE | 0-10% |
| Helianthus Annuus (Sunflower) Seed | 0-10% |
| Oil | |
| Alteromonas Ferment Extract | 0-2% |
| Sodium Acetylated Hyaluronate | 0-0.1% |
| Sodium Hyaluronate | 0-0.1% |
| Sodium Hyaluronate Crosspolymer | 0-0.1% |
| Hydrolyzed Sodium Hyaluronate | 0-0.1% |
| Ethylhexylglycerin | 0.00075% |
| Sodium Acrylates Copolymer | 0-5% |
| Centella Asiatica Extract | 0-5% |
| Cetyl Alcohol | 0-5% |
| Hydroxyacetophenone | 0-5% |
| 1,2-Hexanediol | 0-5% |
| Caprylyl Glycol | 0-5% |
| Sodium Hydroxide | 0-2% |
| Total | 100.0000% |

82. The method of claim 39, wherein the composition is:

| Ingredient | % w/w |
|---|---|
| Water | 40-90% |
| Caprylic/Capric Triglyceride | 2-20% |
| Glycerin | 1-15% |
| Silica | 1-10% |
| Dimethyl Isosorbide | 1-10% |
| Squalane | 0.5-5% |
| Diheptyl Succinate | 0.4-4% |
| Capryloyl Glycerin/Sebacic Acid | 0.5-5% |
| Copolymer | |
| Arachidyl Alcohol | 0.5-5% |
| Microcrystalline Cellulose | 0.2-5% |
| Hydroxyethyl Acrylate/Sodium | 0.2-5% |
| Acryloyldimethyl Taurate Copolymer | |
| Behenyl Alcohol | 0.2-5% |
| Hydroxyacetophenone | 0.2-5% |
| Cetearyl Olivate | 0.2-5% |
| Sortiban Olivate | 0.2-5% |
| Hydrogenated Olive Olil | 0.2-5% |
| Naringenin | 0.2-5% |
| Arachidyl Glucoside | 0.2-2% |
| Lauroyl Lysine | 0.1-2% |
| Pentylene Glycol | 0.1-2% |
| 1,2-Hexanediol | 0.1-2% |
| Caprylyl Glycol | 0.1-2% |
| Olea Europaea (Olive) Fruit Oil | 0.05-1% |
| Disodium Acetyl Glucosamine | 0.02-0.5% |
| Phosphate | |
| Sodium Glucuronate | 0.02-0.5% |
| Phospholipids | 0.02-0.5% |
| Olea Europaea (Olive) Oil | 0.01-0.2% |
| Unsaponifiables | |
| Polysorbate 60 | 0.01-0.2% |
| Sorbitan Isostearate | 0.01-0.2% |
| Magnesium Sulfate | 0.005-0.1% |
| Sodium Benzoate | 0.005-0.1% |
| Sphingolipids | 0.005-0.1% |
| Sodium Acetylated Hyaluronate | 0.005-0.1% |
| Sodium Hyaluronate | 0.005-0.1% |
| Sodium Citrate | 0.0025-0.05% |
| p-Anisic Acid | 0.0015-0.05% |
| Sodium Hyaluronate Crosspolymer | 0.0015-0.05% |
| Hydrolyzed Sodium Hyaluronate | 0.00015-0.005% |
| Acetyl Hexapeptide-8 | 0.00015-0.005% |
| Ethylhexlglycerin | 0.00015-0.005% |
| Tocopherol | 0.000015-0.0005% |
| Sum Total | 100.0000% |

83. The method of claim 39, wherein the composition is:

| Ingredients | % w/w |
|---|---|
| Water | 25-80% |
| Zinc Oxide | 5-40% |
| Caprylic/Capric Triglyceride | 1-20% |
| Dicaprylyl Carbonate | 1-20% |
| C13-15 Alkane | 1-20% |
| Dimethyl Isosorbide | 0.5-10% |
| Propanediol | 0.5-10% |
| Cetearyl Alcohol | 0.25-5% |
| Neopentyl Glycol Dipheptanoate | 0.25-5% |
| Polyhydroxystearic Acid | 0.25-5% |
| Polyglyceryl-3 Polyricinoleate | 0.25-5% |
| Isotearic Acid | 0.25-5% |
| Lecithin | 0.25-5% |
| Octyldodecanol | 0.25-5% |
| Coco-Glucoside | 0.25-5% |
| Trehalose | 0.25-5% |
| Potassium Cetyl Phosphate | 0.25-5% |
| Simmondsia Chinensis (jojoba) Seed Oil | 0.25-5% |
| Citric Acid | 0.1-5% |
| Isododecane | 0.1-5% |
| Cellulose | 0.1-5% |
| Caprylyl Glyceryl Ether | 0.1-5% |
| Bis-Diglyceryl Polyacyladipate-2 | 0.1-5% |
| Octyldodecanol | 0.1-5% |
| Naringenin | 0.1-5% |
| Cellulose Gum | 0.1-5% |
| Disteardimonium Hectorite | 0.1-5% |
| Iron Oxide (CI77492) | 0.2400% |
| Acacia Senegal Gum | 0.05-1% |
| Caprylhydroxamic Acid | 0.05-1% |
| Propylene Carbonate | 0.05-1% |
| Xanthan Gum | 0.05-1% |
| Glycerin | 0.05-1% |
| Sodium Phytate | 0.05-1% |
| Aloe Barbadensis Leaf Juice Powder | 0.005-0.2% |
| Glucose | 0.001-0.05% |
| Iron Oxide (CI77491) | 0.001-0.05% |
| Dunaliella Saline Extract | 0.0005-0.005% |
| Total | 100.0000% |

84. The method of claim 39, wherein the composition is:

| Ingredient | % w/w |
|---|---|
| Water | 40-90% |
| Glycerin | 1-10% |
| Diheptyl Succinate | 1-10% |
| Caprylic/Capric Triglyceride | 1-10% |
| Dimethyl Isosorbide | 0.5-5% |
| Squalane | 0.5-5% |
| C15-19 Alkane | 0.5-5% |
| Arachidyl Alcohol | 0.1-5% |
| Pseudozyma Epicola/Sunflower Seed Oil | 0.1-5% |
| Ferment Extract Filtrate | |
| Propanediol | 0.1-5% |
| Hydroxyethyl Acrylte/Sodium | 0.1-5% |
| Acryloyldimethyl Taurate Copolymer | |
| Xylitylglucoside | 0.1-5% |
| Behenyl Alcohol | 0.1-2.5% |
| Saccharide Isomerate | 0.1-2.5% |
| Caprylyl Glyceryl Ether | 0.1-2.5% |
| Cetearyl Olivate | 0.1-2.5% |
| Sortiban Olivate | 0.1-2.5% |
| Hydrogenated Olive Oil | 0.1-2.5% |
| Anhydroxylitol | 0.1-2.5% |
| Naringenin | 0.1-5% |
| Arachidyl Glucoside | 0.1-2.5% |
| Phospholipids | 0.1-2.5% |
| Phytosterols | 0.1-2.5% |
| Linoleic Acid | 0.1-2.5% |
| Ceramide NP | 0.1-2.5% |
| Propylene Glycol | 0.05-2% |
| Xylitol | 0.05-2% |
| Caprylhydroxamic Acid | 0.05-2% |
| Olea Europaea (Olive) Fruit Oil | 0.05-2% |
| Capryloyl Glycerin/Sebacic Acid | 0.01-2% |
| Copolymer | |
| Olea Europaea (Olive) Oil | 0.01-1% |
| Unsaponifiables | |
| Glucose | 0.01-1% |
| Polysorbate 60 | 0.01-1% |
| Sorbitan Isostearate | 0.01-1% |
| Hydrogenated Lecithin | 0.01-1% |
| Glyceryl Stearate | 0.005-0.5% |
| Citric Acid | 0.005-0.5% |
| Sodium Citrate | 0.001-0.5% |
| Ceramide NS | 0.001-0.5% |
| Ceramide AG | 0.001-0.5% |
| Ceramide AP | 0.001-0.5% |
| Cholesterol | 0.001-0.5% |
| Ceramide BOP | 0.00001-0.005% |
| Total | 100.000% |

85. The method of claim 39, wherein the composition is:

| Ingredients | % w/w |
|---|---|
| Water | 40-80% |
| Gluconolactone | 2.5-25% |
| Squalane | 1-10% |
| Glycerin | 1-10% |
| Propanediol | 1-10% |
| Hemisqualane | 1-10% |
| Neopentyl Glycol Diheptanoate | 0.5-5% |
| Dimethyl Isosorbide | 0.5-5% |
| Sodium Polyacryloyldimethyl Taurate | 0.5-5% |
| Polyglyceryl-3Methylglucose Distearate | 0.5-5% |
| Isododecane | 0.5-5% |
| Hydroxyethyl Acrylate/Sodium Acryloyl | 0.1-2.5% |
| Dimethyl Taurate Copolymer | |
| Helianthus Annuus Seed Oil | 0.1-2.5% |
| Sodium Hydroxide | 0.1-2.5% |
| Hydroxyacetophenone | 0.1-2.5% |
| Naringenin | 0.1-5% |
| Inulin Lauryl Carbamate | 0.1-5% |
| Persea Gratissima (Avocado) Fruit Extract | 0.05-2.5% |
| Maltodextrin | 0.05-2.5% |
| Caprylyl Glycol | 0.05-2.5% |
| 1,2-Hexanediol | 0.05-2.5% |
| Hydrolyzed Jojoba Esters | 0.05-2.5% |
| Pentylene Glycol | 0.05-2.5% |
| Sodium Acetylated Hyaluronate | 0.005-0.25% |
| Sodium Hyaluronate | 0.0025-0.25% |
| Capsicum Annuum Fruit Extract | 0.0015-0.05% |
| Sodium Hyaluronate Crosspolymer | 0.0015-0.05% |
| Hydrolyzed Sodium Hyaluronate | 0.00015-0.005% |
| Ethylhexlglycerin | 0.0001-0.01% |
| Total | 100% |

86. A method of modulating expression of one or more genes by administration of a composition comprising naringenin, wherein at least one of the genes is related to a topical condition.

87. The method of claim 86, wherein the one or more genes are selected from a group consisting of: LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, MIR205, CCL11, CSF2, IL23A, IL11, BMP6, IL13RA2, C2CD4A, NR4A2, C2CD4B, FGF2, FABP4, GDF15, THBS1, HSPA1A, PTGS2, DKK1, and any combination thereof.

88. The method of claim 86, wherein the administration of the composition leads to up-regulation of LCE5A, KRT77, ALOXE3, KRT1, HPGD, MUCL1, CASP14, WFDC12, KRT10, DNASE1L2, IL37, SERPINB12, HAL, UPK1A, CALML5, LCE2B, and/or MIR205.

89. The method of claim 86, wherein the administration of the composition leads to down-regulation of CCL11, CSF2, IL23A, IL11, BMP6, IL13RA2, C2CD4A, NR4A2, C2CD4B, FGF2, FABP4, GDF15, THBS1, HSPA1A, PTGS2, and/or DKK1.

90. The method of claim 86, wherein the administration of the composition enhances anti-inflammatory activity.

91. The method of claim 86, wherein the administration of the composition enhances the skin barrier strength and/or maintenance of skin barrier lipids.

92. The method of claim 86, wherein the administration of the composition enhances cell turnover and cell recycling.

93. The method of claim 92, wherein the enhancement of cell turnover and cell recycling has an antiaging effect.

94. The method of claim 93, wherein the antiaging effect leads to prevention of formation of fine lines and/or wrinkles in the skin.

95. The method of claim 90, wherein the administration of composition leads to downregulation of CSF2, IL23A, IL11, IL13RA2, C2CD4A, NR4A2, FABP4, GDF15, and/or PTGS2.

96. The method of claim 90, wherein the administration of composition leads to upregulation of HPGD.

97. The method of claim 91, wherein the administration of composition leads to upregulation of LCE5A, KRT77, ALOXE3, KRT1, CASP14, KRT10, and/or LCE2B.

98. The methods of claim 92 or 93, wherein the administration of composition leads to upregulation of DNASE1L2, SERPINB12, UPK1A, CALML5 and/or MIR205.

99. The methods of claim 92 or 93, wherein the administration of composition leads to downregulation of BMP6 and/or HSPA1A.

100. The method of claim 86, wherein the administration of the composition results in at least about thirty (30) fold up-regulation in expression and/or activity of LCE5A.

101. The method of claim 86, wherein the administration of the composition results in at least about ten (10) fold up-regulation in expression and/or activity of KRT77.

102. The method of claim 86, wherein the administration of the composition results in at least about ten (10) fold up-regulation in expression and/or activity of ALOXE3.

103. The method of claim 86, wherein the administration of the composition results in at least about five (5) fold up-regulation in expression and/or activity of KRT1.

104. The method of claim 86, wherein the administration of the composition results in at least about ten (10) fold up-regulation in expression and/or activity of HPGD.

105. The method of claim 86, wherein the administration of the composition results in at least about ten (10) fold up-regulation in expression and/or activity of MUCL1.

106. The method of claim 86, wherein the administration of the composition results in at least about ten (10) fold up-regulation in expression and/or activity of CASP14.

107. The method of claim 86, wherein the administration of the composition results in at least about ten (10) fold up-regulation in expression and/or activity of WFDC12.

108. The method of claim 86, wherein the administration of the composition results in at least about five (5) fold up-regulation in expression and/or activity of KRT10.

109. The method of claim 86, wherein the administration of the composition results in at least about five (5) fold up-regulation in expression and/or activity of DNASE1L2.

110. The method of claim 86, wherein the administration of the composition results in at least about ten (10) fold up-regulation in expression and/or activity of IL37.

111. The method of claim 86, wherein the administration of the composition results in at least about ten (10) fold up-regulation in expression and/or activity of SERPINB12.

112. The method of claim 86, wherein the administration of the composition results in at least about five (5) fold up-regulation in expression and/or activity of HAL.

113. The method of claim 86, wherein the administration of the composition results in at least about ten (10) fold up-regulation in expression and/or activity of UPK1A.

114. The method of claim 86, wherein the administration of the composition results in at least about five (5) fold up-regulation in expression and/or activity of CALML5.

115. The method of claim 86, wherein the administration of the composition results in at least about five (5) fold up-regulation in expression and/or activity of LCE2B.

116. The method of claim 86, wherein the administration of the composition results in at least about five (5) fold up-regulation in expression and/or activity of MIR205.

117. The method of claim 86, wherein the administration of composition results in at least about twenty (20) fold downregulation of expression and/or activity of CCL11.

118. The method of claim 86, wherein the administration of composition results in at least about fifteen (15) fold downregulation of expression and/or activity of CSF2.

119. The method of claim 86, wherein the administration of composition results in at least about fifteen (15) fold downregulation of expression and/or activity of IL23A.

120. The method of claim 86, wherein the administration of composition results in at least about ten (10) fold downregulation of expression and/or activity of IL11.

121. The method of claim 86, wherein the administration of composition results in at least about ten (10) fold downregulation of expression and/or activity of BMP6.

122. The method of claim 86, wherein the administration of composition results in at least about ten (10) fold downregulation of expression and/or activity of IL3RA2.

123. The method of claim 86, wherein the administration of composition results in at least about ten (10) fold downregulation of expression and/or activity of C2CD4A.

124. The method of claim 86, wherein the administration of composition results in at least about ten (10) fold downregulation of expression and/or activity of NR4A2.

125. The method of claim 86, wherein the administration of composition results in at least about five (5) fold downregulation of expression and/or activity of C2CD4B.

126. The method of claim 86, wherein the administration of composition results in at least about five (5) fold downregulation of expression and/or activity of FGF2.

127. The method of claim 86, wherein the administration of composition results in at least about five (5) fold downregulation of expression and/or activity of FABP4.

128. The method of claim 86, wherein the administration of composition results in at least about five (5) fold downregulation of expression and/or activity of GDF15.

129. The method of claim 86, wherein the administration of composition results in at least about five (5) fold downregulation of expression and/or activity of THBS1.

130. The method of claim 86, wherein the administration of composition results in at least about five (5) fold downregulation of expression and/or activity of HSPA1A.

131. The method of claim 86, wherein the administration of composition results in at least about five (5) fold downregulation of expression and/or activity of PTGS2.

132. The method of claim 86, wherein the administration of composition results in at least about five (5) fold downregulation of expression and/or activity of DKK1.

133. The method of claim 86, wherein the administration of the composition to a subject leads to the treatment of prevention of a topical condition of the subject.

134. The method of claim 86, wherein the administration of composition alters one or more of a function related with the modulated genes, wherein the one or more function is selected from the group consisting of: (i) cell turnover, (ii) inflammation, (iii) barrier support, and (iv) any combination thereof.

135. The method of claim 86, wherein the composition further comprises a solvent.

136. The method of claim 135, wherein the solvent is selected from a group consisting of: dimethyl isosorbide, ethoxydiglycol, isopropyl lauryl sarcosinate, pentylene glycol, propanediol, 1,2-hexanediol, and/or butylene glycol.
